SEQUENCE LISTING (53 sequences)

SEQ ID NO: 1 - RNA, 25 nt, Artificial
  siRNA +strand; Nucleotides at residues 1-23 are ribonucleotides.
ucuacagcuucugauaaucaaccaa

SEQ ID NO: 2 - RNA, 27 nt, Artificial
  siRNA -strand; Nucleotides at residues 1-27 are ribonucleotides.
uugguugauuaucagaagcuguagaaa

SEQ ID NO: 3 - RNA, 25 nt, Artificial
  siRNA +strand; Nucleotides at residues 1-23 are ribonucleotides.
cuugcuaccaguauuuagaagccaa

SEQ ID NO: 4 - RNA, 27 nt, Artificial
  siRNA -strand; Nucleotides at residues 1-27 are ribonucleotides.
uuggcuucuaaauacugguagcaagau

SEQ ID NO: 5 - RNA, 25 nt, Artificial
  siRNA +strand; Nucleotides at residues 1-23 are ribonucleotides.
accuuccaugucaagauucagcuag

SEQ ID NO: 6 - RNA, 27 nt, Artificial
  siRNA -strand; Nucleotides at residues 1-27 are ribonucleotides.
cuagcugaaucuugacauggaagguuu

SEQ ID NO: 7 - DNA, 25 bp, Artificial
  siRNA +strand; Nucleotides at residues 1-23 are ribonucleotides.
aaaucauguuggagaucucuauugt

SEQ ID NO: 8 - RNA, 27 nt, Artificial
  siRNA -strand; Nucleotides at residues 1-27 are ribonucleotides.
acaauagagaucuccaacaugauuuca

SEQ ID NO: 9 - DNA, 25 bp, Artificial
  siRNA +strand; Nucleotides at residues 1-23 are ribonucleotides.
aaggaauccaguugaauacaauuct

SEQ ID NO: 10 - RNA, 27 nt, Artificial
  siRNA -strand; Nucleotides at residues 1-27 are ribonucleotides.
agaauuguauucaacuggauuccuugu

SEQ ID NO: 11 - DNA, 25 bp, Artificial
  siRNA +strand; Nucleotides at residues 1-23 are ribonucleotides.
aguaugaagucaucgguaauuacta

SEQ ID NO: 12 - RNA, 27 nt, Artificial
  siRNA -strand; Nucleotides at residues 1-27 are ribonucleotides.
uaguaauuaccgaugacuucauacuga

SEQ ID NO: 13 - RNA, 25 nt, Artificial
  siRNA +strand; Nucleotides at residues 1-23 are ribonucleotides.
cgguuugagaauguuccuauaauaa

SEQ ID NO: 14 - RNA, 27 nt, Artificial
  siRNA -strand; Nucleotides at residues 1-27 are ribonucleotides.
uuauuauaggaacauucucaaaccgca

SEQ ID NO: 15 - RNA, 25 nt, Artificial
  siRNA +strand; Nucleotides at residues 1-23 are ribonucleotides.
gagccuuuaguuuucauccauguac

SEQ ID NO: 16 - RNA, 27 nt, Artificial
  siRNA -strand; Nucleotides at residues 1-27 are ribonucleotides.
guacauggaugaaaacuaaaggcucga

SEQ ID NO: 17 - DNA, 25 bp, Artificial
  siRNA +strand; Nucleotides at residues 1-23 are ribonucleotides.
agcugagauguuccuuaguaaucat

SEQ ID NO: 18 - RNA, 27 nt, Artificial
  siRNA -strand; Nucleotides at residues 1-27 are ribonucleotides.
augauuacuaaggaacaucucagcugu

SEQ ID NO: 19 - DNA, 25 bp, Artificial
  siRNA +strand; Nucleotides at residues 1-23 are ribonucleotides.
ggcucagcugauaaacaugaauctg

SEQ ID NO: 20 - RNA, 27 nt, Artificial
  siRNA -strand; Nucleotides at residues 1-27 are ribonucleotides.
cagauucauguuuaucagcugagccaa

SEQ ID NO: 21 - DNA, 25 bp, Artificial
  siRNA +strand; Nucleotides at residues 1-23 are ribonucleotides.
gaauggaaauguauugguaaagcta

SEQ ID NO: 22 - RNA, 27 nt, Artificial
  siRNA -strand; Nucleotides at residues 1-27 are ribonucleotides.
uagcuuuaccaauacauuuccauuccc

SEQ ID NO: 23 - DNA, 25 bp, Artificial
  siRNA +strand; Nucleotides at residues 1-23 are ribonucleotides.
agucugaauggaaggauacuaauta

SEQ ID NO: 24 - RNA, 27 nt, Artificial
  siRNA -strand; Nucleotides at residues 1-27 are ribonucleotides.
uaauuaguauccuuccauucagacucg

SEQ ID NO: 25 - RNA, 25 nt, Artificial
  siRNA +strand; Nucleotides at residues 1-23 are ribonucleotides.
guucugaagucuaagauauuuuuca

SEQ ID NO: 26 - RNA, 27 nt, Artificial
  siRNA -strand; Nucleotides at residues 1-27 are ribonucleotides.
ugaaaaauaucuuagacuucagaacag

SEQ ID NO: 27 - RNA, 25 nt, Artificial
  siRNA +strand; Nucleotides at residues 1-23 are ribonucleotides.
aacuacaccuggauuuuuccaaaca

SEQ ID NO: 28 - RNA, 27 nt, Artificial
  siRNA -strand; Nucleotides at residues 1-27 are ribonucleotides.
uguuuggaaaaauccagguguaguugc

SEQ ID NO: 29 - DNA, 25 bp, Artificial
  siRNA +strand; Nucleotides at residues 1-23 are ribonucleotides.
agugauguuuggaaucgaucaugct

SEQ ID NO: 30 - RNA, 27 nt, Artificial
  siRNA -strand; Nucleotides at residues 1-27 are ribonucleotides.
ugucacuacaaaccuuagcuaguacga

SEQ ID NO: 31 - RNA, 25 nt, Artificial
  siRNA +strand; Nucleotides at residues 1-23 are ribonucleotides.
gagcuuuuuggaaggauaauucuga

SEQ ID NO: 32 - RNA, 27 nt, Artificial
  siRNA -strand; Nucleotides at residues 1-27 are ribonucleotides.
ucagaauuauccuuccaaaaagcucaa

SEQ ID NO: 33 - DNA, 25 bp, Artificial
  siRNA +strand; Nucleotides at residues 1-23 are ribonucleotides.
ggacagugguaaagagcugaaaatt

SEQ ID NO: 34 - RNA, 27 nt, Artificial
  siRNA -strand; Nucleotides at residues 1-27 are ribonucleotides.
aauuuucagcucuuuaccacuguccaa

SEQ ID NO: 35 - DNA, 25 bp, Artificial
  siRNA +strand; Nucleotides at residues 1-23 are ribonucleotides.
ggucaagauguacugcuauuuautg

SEQ ID NO: 36 - RNA, 27 nt, Artificial
  siRNA -strand; Nucleotides at residues 1-27 are ribonucleotides.
caauaaauagcaguacaucuugaccuu

SEQ ID NO: 37 - RNA, 26 nt, Artificial
  Scrambled Negative Control DsiRNA; Nucleotides at residues 1-23 are ribonucleotides; n = 5′ phosphorylated
  misc_feature (1)..(1): n is a, c, g, or u
ncuuccucucuuucucucccuuguga

SEQ ID NO: 38 - RNA, 27 nt, Artificial
  Scrambled Negative Control DsiRNA; Nucleotides at residues 1-27 are ribonucleotides.
ucacaagggagagaaagagaggaagga

SEQ ID NO: 39 - DNA, 28 bp, Artificial
  TYE 563 Transfection Control DsiRNA; Nucleotides at residues 3-25 are ribonucleotides; n = 5′ TYE563 modification
  misc_feature (1)..(1): n is a, c, g, t or u
ntccuuccucucuuucucucccuuguga

SEQ ID NO: 40 - DNA, 28 bp, Artificial
  TYE 563 Transfection Control DsiRNA; Nucleotides at residues 3-25 are ribonucleotides; n = 5′ TYE563 modification
  misc_feature (1)..(1): n is a, c, g, t or u
ntcacaagggagagaaagagaggaagga

SEQ ID NO: 41 - DNA, 31 bp, Artificial
  Single stranded DNA oligonucleotide
taatacgactcactatagggcgaaaacgccc

SEQ ID NO: 42 - DNA, 88 bp, Artificial
  Single stranded DNA oligonucleotide
actatagggcgaaaacgccccattttattatgtatggaaaagttatgtgcatgttgtagacggttgtaattcatcaacttgtatgatg

SEQ ID NO: 43 - DNA, 85 bp, Artificial
  Single stranded DNA oligonucleotide
aacaccattaacaatagttgtacattcgactcttgttgctctattacgtttgtaacacatcatacaagttgatgaattacaaccg

SEQ ID NO: 44 - DNA, 28 bp, Artificial
  Single stranded DNA oligonucleotide
aacaccattaacaatagttgtacattcg

SEQ ID NO: 45 - DNA, 90 bp, Artificial
  Single stranded DNA oligonucleotide
actatagggcgaaaacgcccgaattttatcatgactgaactgtttttgtgtcccaaaaatagtgctacattaggaaaataattaagatta

SEQ ID NO: 46 - DNA, 90 bp, Artificial
  Single stranded DNA oligonucleotide
ccctcctgataagtaaaccctagtactaaataacatattaactccaattcagccgtaatcttaattattttcctaatgtagcactatttt

SEQ ID NO: 47 - DNA, 23 bp, Artificial
  Single stranded DNA oligonucleotide
ccctcctgataagtaaaccctag

SEQ ID NO: 48 - DNA, 156 bp, Artificial
  Double stranded DNA, PCR product
taatacgactcactatagggcgaaaacgccccattttattatgtatggaaaagttatgtgcatgttgtagacggttgtaattcatcaacttgtatgatgtgttacaaacgtaatagagcaacaagagtcgaatgtacaactattgttaatggtgtt

SEQ ID NO: 49 - DNA, 176 bp, Artificial
  Double stranded DNA, PCR product
taatacgactcactatagggcgaaaacgcccactatagggcgaaaacgcccgaattttatcatgactgaactgtttttgtgtcccaaaaatagtgctacattaggaaaataattaagattacggctgaattggagttaatatgttatttagtactagggtttacttatcaggaggg

SEQ ID NO: 50 - RNA, 139 nt, Artificial
  In vitro transcribed RNA
gggcgaaaacgccccauuuuauuauguauggaaaaguuaugugcauguuguagacgguuguaauucaucaacuuguaugauguguuacaaacguaauagagcaacaagagucgaauguacaacuauuguuaaugguguu

SEQ ID NO: 51 - RNA, 139 nt, Artificial
  In vitro transcribed RNA
gggcgaaaacgcccgaauuuuaucaugacugaacuguuuuugugucccaaaaauagugcuacauuaggaaaauaauuaagauuacggcugaauuggaguuaauauguuauuuaguacuaggguuuacuuaucaggaggg

SEQ ID NO: 52 - DNA, 22 bp, Artificial
  LNA7472-7493; Nucleotides at 1-6, 8-10, 12, 14-16, 18-22 are locked nucleic acid bases.
tacaagttgatgaattacaacc

SEQ ID NO: 53 - DNA, 19 bp, Artificial
  Scrambled LNA; Nucleotides at 1-3, 6, 9, 12, 14, 17-19 are locked nucleic acid bases.
gcggcacgttgcgagtact

(19) United States
(12) Patent Application Publication
(10) Pub. No.: US 2025/0263697 A1
(43) Pub. Date: Aug. 21, 2025

(54) COMPOSITIONS AND METHODS OF MODULATING RNA AND PROTEIN INTERACTIONS

(71) Applicant: The Regents of the University of California, Oakland, CA (US)

(72) Inventors: Eugene Yeo, La Jolla, CA (US); Shengnan Xiang, La Jolla, CA (US)

(21) Appl. No.: 18/565,689

(22) PCT Filed: Jun. 1, 2022
(86) PCT No.: PCT/US2022/031773

§ 371 Date: Nov. 30, 2023

Related U.S. Application Data
(60) Provisional application No. 63/196,005, filed on Jun. 2, 2021.

(51) Int. Cl. C12N 15/113

(52) U.S. Cl. CPC C12N 15/113; C12N 2310/14; C12N 2310/315; C12N 2310/321; C12N 2310/322; C12N 2310/3231

(57) ABSTRACT

Provided are compositions and methods of treating Coronavirus disease 2019 (COVID-19) in a subject, the method including administering to the subject a therapeutically effective amount of a composition comprising an exogenous nucleic acid and delivering the exogenous nucleic acid into a cell, wherein the exogenous nucleic acid comprises an antisense oligonucleotide, a small interfering RNA (siRNA), or locked nucleic acid, and wherein the exogenous nucleic acid binds to a target RNA and modulates gene expression of the target RNA, thereby treating Coronavirus disease 2019 (COVID-19) in the subject.

### CROSS-REFERENCE TO RELATED APPLICATIONS

This application is a National Stage Application under 35 U.S.C. § 371 and claims the benefit of International Application No. PCT/US2022/031773, filed Jun. 1, 2022, which claims priority to U.S. Provisional Patent Application No. 63/196,005, filed on Jun. 2, 2021. The disclosure of the prior application is considered part of the disclosure of this application, and is incorporated herein by reference in its entirety.

### SEQUENCE LISTING

This application contains a Sequence Listing that has been submitted electronically as an ASCII text file named “15670-0353US1_UPDATEDSL_ST25.txt.” The ASCII text file, created on Dec. 7, 2023, is 13,535 bytes in size. The material in the ASCII text file is hereby incorporated by reference in its entirety.

### BACKGROUND

COVID-19 is caused by the novel severe acute respiratory syndrome coronavirus 2 (SARS-CoV-2), a positive-sense single-stranded (+ss) RNA virus. The viral genome encodes 29 proteins, which include the four structural proteins, membrane or matrix (M), nucleocapsid (N), envelope (E), and spike (S) proteins. In addition, there are 16 non-structural proteins, NSP1-16, and 9 accessory proteins ORF3a-ORF10, though the expression of some of the accessory factors are still debated. Identification of conserved viral RNA processes, viral protein-host RNA interactions, and understanding how the virus hijacks these processes will enable the discovery of new antiviral targets and strategies.

Recent transcriptome-wide and proteome-wide studies in viral protein-host protein interactions, viral protein and RNA interactions with host proteins, and viral RNA-host RNA interactions contribute to the understanding of host-virus interactions in SARS-CoV-2 infection. A recent study on the protein interactome with viral RNA shows that many of the SARS-CoV-2 proteins are RNA binding proteins that bind to its own RNA genome and mRNA transcripts. As a (+ss) RNA virus, SARS-CoV-2 proteins are also found to associate with several host RNA binding proteins (RBPs), suggesting a possibility that SARS-CoV-2 proteins interact with the host transcriptome to a greater degree than previously anticipated. For example, it has been shown that NSP1 binds to the mRNA entry site on the host ribosomal RNA to inhibit host translation. SARS-CoV-2 nucleocapsid protein interactome comprises many host RNA processing machinery proteins and stress granule proteins, suggesting a potential role in interfering with host RNA processing and driving stress granule formation. One study showed that SARS-CoV-2 proteins bind to ˜140 host transcripts. Focusing on non-coding RNAs, SARSCoV-2 NSP16 was found to bind to U1/U2 snRNA to interfere with splicing, while NSP8 and NSP9 bind to the signal recognition ribonucleoprotein 7SK to block protein trafficking. However, knowledge of how viral and host coding RNAs interact with viral proteins and the functional implications of these interactions remain limited. Thus, a comprehensive interrogation of SARS-CoV-2 viral protein-RNA interactions is still needed to gain insights into viral RNA processing and how the virus hijacks host cellular machinery for its replication while simultaneously suppressing host gene expression.

### SUMMARY

Provided herein are pharmaceutical compositions comprising an inhibitory nucleic acid that is complementary to a portion of a target RNA. In some embodiments, the target RNA comprises a portion of the SARS-CoV-2 genome. In some embodiments, the target RNA comprises a portion of human RNA. In some embodiments, the target RNA comprises human mRNA. In some embodiments, the target RNA is associated with a portion of the 5′ untranslated region (UTR) of the target RNA. In some embodiments, the target RNA is associated with a portion of the coding region of the target RNA. In some embodiments, the target RNA is associated with a portion of the 3′ untranslated region of the target RNA.

In some embodiments, a SARS-CoV-2 protein selected from the group consisting of NSP2, NSP3, NSP5, NSP6, NSP7, NSP9, NSP12, NSP14, NSP15, ORF3b, ORF7b and ORF9c is capable of binding to a portion of the target RNA. In some embodiments, the target RNA is associated with a gene selected from the group consisting of RPN1, UGGT1, OST4, NDUFA4, HSPA5, PSMD13, LAPTM4A, LAMP1, C1QBP, and LDHB.

In some embodiments, the inhibitory nucleic acid comprises one or more small interfering RNAs (siRNA). In some embodiments, the one or more siRNAs comprise at least one sequence selected from the group consisting of SEQ ID NOs: 1-40. In some embodiments, the inhibitory nucleic acid comprises one or more antisense oligonucleotides. In some embodiments, the inhibitory nucleic acid comprises one or more locked nucleic acids (LNA). In some embodiments, the one or more LNAs comprises a sequence selected from the group consisting of SEQ ID NOs: 52 and 53. In some embodiments, the inhibitory nucleic acid comprises one or more phosphorothioate substitutions. In some embodiments, the inhibitory nucleic acid comprises one or more 2′-O-methyl modifications. In some embodiments, the inhibitory nucleic acid comprises one or more 2′-O-methoxyethyl modifications. In some embodiments, the inhibitory nucleic acid comprises one or more 2′-fluoro modifications.

In some embodiments, the one or more siRNAs, antisense oligonucleotides, or locked nucleic acids, are a plurality, wherein each siRNA, antisense oligonucleotide, or locked nucleic acid comprises the same sequence as the other siRNAs, antisense oligonucleotides, or locked nucleic acids in the plurality. In some embodiments, the one or more siRNAs, antisense oligonucleotides, or locked nucleic acids contain a mixture of siRNAs, antisense oligonucleotides, or locked nucleic acids comprising at least two different sequences.

In some embodiments, the LNA is enclosed within a vector. In some embodiments, the vector is a lentivirus. In some embodiments, the vector is an adenovirus. In some embodiments, the vector is an adeno-associated viral vector (AAV). In some embodiments, the vector is a liposome.

Also provided herein are methods of treating Coronavirus disease 2019 (COVID-19) in a subject, the method comprising: administering to the subject a therapeutically effective amount of any one of the pharmaceutical compositions described herein, thereby treating Coronavirus disease 2019 (COVID-19) in the subject. In some embodiments, the therapeutically effective amount of the pharmaceutical composition decreases target RNA protein expression.

Also provided herein are methods of reducing risk of SARS-CoV2 infection in a subject, the method comprising: administering to the subject a therapeutically effective amount of any one of the pharmaceutical compositions described herein, thereby reducing risk of SARS-CoV2 infection in the subject.

Also provided herein are inhibitory nucleic acids which is or comprises SEQ ID NOs: 1-40, wherein the inhibitory nucleic acid comprises a siRNA.

Also provided herein are inhibitory nucleic acids which is or comprises SEQ ID NOs: 52 or 53, wherein the inhibitory nucleic acid comprises a LNA.

### BRIEF DESCRIPTION OF DRAWINGS

FIG. 1A is an exemplary schematic showing eCLIP performed on SARS-CoV-2 proteins in virus infected Vero E6 cells. Proteins in infected cells were UV crosslinked to bound transcripts, which were immunoprecipitated (IP) with antibodies that recognize NSP8 (primase), NSP12 (RNA dependent RNA polymerase, RdRp) and N (nucleocapsid) proteins. Protein-RNA IP product and Input lysate were resolved by SDS-PAGE and membrane transferred, followed by band excision at the estimated protein size to 75 kDa above in both IP and Input lanes. Excised bands were subsequently purified, and library barcoded for Illumina sequencing.

FIG. 1B shows mean fold change of eCLIP read density mapped to the positive sense SARS-CoV-2 genome in immunoprecipitated (IP) compared to input samples. Mean was taken from n=2 independent biological samples.

FIG. 1C shows NSP12 eCLIP zoomed into yellow highlighted regions in FIG. 1B. Top row, NSP12 eCLIP; bottom row, SHAPE Shannon entropy with a sliding median of 55 nt. Shaded region in bottom row is partitioned at the global median entropy.

FIG. 1D shows correlation between normalized SHAPE entropy and normalized log 2 (Fold Change) of IP over INPUT eCLIP read density i.e. eCLIP enrichment for NSP12 (left), NSP8 (middle) and N (right). R, Pearson's coefficient.

FIG. 1E shows secondary structure of the NSP12 eCLIP peak region from position 7412-7545.

FIG. 1F shows results from filter-binding assay showing RNA crosslinked to NSP12 on the nitrocellulose layer, and the free RNA on the nylon layer.

FIG. 1G shows fraction of RNA bound to NSP12 from filter binding assay, using hairpin RNA from position 7414-7555 and scrambled RNA as negative control.

FIG. 1H shows correlation matrix of mean fold change of eCLIP read density: Bottom left panels, 2D density plots; diagonal, density plot corresponding to samples in bottom labels; top right panels, Pearson's coefficient between samples.

FIG. 1I shows a locked nucleic acid (LNA) designed to be complementary to the hairpin RNA from position 7414-7555.

FIG. 1J shows complementary LNA targeting SARS-CoV-2 genome shown to hybridize and disrupt the structure of the central hairpin as predicted by NUPACK software.

FIG. 1K shows immunofluorescence microscopy images showing fraction of infected cells as stained by SARS-CoV-2 nucleocapsid proteins (green), with cell nucleus stained with DAPI (green).

FIG. 1L shows a bar plot showing mean relative fluorescence intensities of cells from FIG. 1K, dots represent segmented individual cells (mean±s.e.m.*p<0.01, two-tailed t-test).

FIG. 2A shows a bar plot showing number of all genes, number of all peaks, number of coding genes and number of peaks mapping to coding genes from n=2 biologically independent replicates of NSP12, NSP8 and N eCLIP of SARS-CoV-2 infected cells. Target genes have at least one reproducible peak (by IDR12) associated with each protein.

FIG. 2B shows a stacked bar plot showing TPM of reads mapped to the Vero E6 genome or SARS-CoV-2 genome in each of NSP12, NSP8 and N eCLIP.

FIG. 2C shows a Venn diagram showing number of African Green Monkey (host) genes targeted by NSP8 and NSP12.

FIG. 2D shows a Violin plot showing the distribution of Log 2FoldChange in transcript levels in Vero E6 cells infected by SARS-CoV-2, for significantly differentially expressed genes (adjusted P<0.05). Kolmogorov-Smirnov test p-values between eCLIP targets of NSP12 and NSP8 versus all differentially expressed genes are indicated above the plot.

FIG. 2E shows Top 25 Enriched Gene Ontology (GO) processes (adjusted p<0.01) for NSP12 target host genes. Box plot indicates quartiles of differential expression (log 2(Fold Change)) of target genes (grey dots).

FIG. 3A shows an exemplary schematic showing SARS-CoV-2 proteins individually tagged and expressed in human lung epithelial cells BEAS-2B to assay with eCLIP.

FIG. 3B shows a bar plot indicating number of all genes, number of all peaks, number of coding genes and number of coding peaks found to interact with each protein from n=2 biologically independent experiments. In addition to SARS-CoV-2 proteins, ENCODE eCLIP data for example human RNA-binding proteins (hRBPs) are included for comparison. Target genes have at least one reproducible peak (by IDR) associated with each protein.

FIG. 3C shows a clustermap showing unique host coding genes (columns) targeted by each SARS-CoV-2 protein (rows).

FIG. 3D shows example genome browser tracks for NSP3, NSP12, N and NSP2 mapping to DYNCH1, TUSC3, CXCL5 and NAP1L4 respectively.

FIGS. 3E-3F show Western blots showing viral (pink background) and human (blue background) proteins enriched via CLASP (FIG. 3E) and RIC (FIG. 3F), with total cell lysate showed in input column (IN).

FIG. 3G shows Enriched Gene Ontology (GO) processes (adjusted p-value<10-5) of unique eCLIP target coding genes for various SARS-CoV-2 proteins.

FIG. 3H shows example sequence logos generated from all IDR peak reads for each SARS-CoV-2 eCLIP, with p-value indicated above each logo.

FIG. 4A shows a stacked bar plot showing fraction of reproducible peaks (by IDR14) mapping to different regions of coding genes. 3ss, 3′ splice site; 3utr, 3′ untranslated region (UTR), 5ss, 5′ splice site; 5utr, 5′ UTR; CDS, coding sequence.

FIG. 4B shows a clustermap showing read density of target RNA by each SARS-CoV-2 protein scaled to a metagene profile containing 5′ UTR, CDS and 3′ UTR regions.

FIG. 4C shows an exemplary schematic showing the Renilla-MS2 and Firefly dual luciferase reporter constructs, where individual SARS-CoV-2 proteins fused to MCP are recruited to the Renillia-MS2 mRNA.

FIGS. 4D-4E show bar plots showing luciferase reporter activity ratios (FIG. 4D) and reporter RT-qPCR ratios (FIG. 4E) for the indicated coexpressed SARS-CoV-2 protein, known human regulators of RNA stability (CNOT7, BOLL) and negative control (FLAG peptide). Ratios are normalized to the negative control (mean±s.e.m., n=3 biologically independent replicate transfections; * p<0.05, ** p<0.005, *** p<0.0005, **** p<0.0001, two-tailed multiple t-test; ns, not significant).

FIG. 4F shows a bar plot showing the fold change of luciferase activity ratio and RT-qPCR ratio (mean±s.e.m, n=3; * p<0.05, *** p<0.001, two-tailed Welch's t-test).

FIG. 4G shows a cumulative distributive plot (CDF) of log 2(Fold Change) of gene expression in HEK293T cells transfected with a plasmid overexpressing NSP12 versus an empty vector plasmid. KS test p values indicate significance of difference in differential expression of NSP12 target genes versus non-eCLIP target genes.

FIG. 4H shows Enriched Gene Ontology (GO) processes (adjusted p<10-4) of NSP12 target genes, with box plots indicating quartiles of differential expression (log 2(Fold Change)) of target genes (black dots).

FIG. 4I shows a CDF plot of Δ log 2(Fold Change) of polysomal mRNA levels in BEAS-2B cells nucleofected with a plasmid overexpressing ORF9c versus an empty vector plasmid. KS test p values indicate significance of difference in differential expression of ORF9c target genes versus non-eCLIP target genes.

FIG. 4J shows Enriched BioPlanet pathways (adjusted p<0.01) of ORF9c target genes, with box plots indicating quartiles of differential expression (Δ log 2(Fold Change) of polysomal mRNA levels) of target genes (black dots).

FIG. 4K shows immunofluorescence images (40×) of SARS-CoV-2 infected A549-ACE2 cells stained for SARS-CoV-2 NSP8 (red), endogenous genes (green), DNA content (blue).

FIG. 4L shows a heat map showing infection rate as measured by the integrated intensity of immunofluorescence staining of SARS-CoV-2 nucleocapsid protein in human iPSC derived lung organoid cells. Cells were treated with siRNAs targeting different host genes prior to viral infection by three different variants of SARS-CoV-2. Significant differences in infection rates were given by two-tailed t-test, * p<0.05, **p<0.01, ns, not significant, as compared to scrambled siRNA control for n=3 biologically independent samples.

FIG. 5A shows a pie charts showing distribution of eCLIP peaks across different coding RNA regions for NSP2, NSP5, NSP7 and NSP9. Genomic content and exonic content are based on the hg19 human reference genome.

FIG. 5B shows Jaccard index similarity of NSP9 target genes as compared with all 223 ENCODE RBP datasets.

FIG. 5C shows metadensity of eCLIP reads truncation sites averaged across all RNA targets by SARS-CoV-2NSP2, NSP5, NSP7 and NSP9, and U2AF1/2 from the ENCODE consortium, zoomed into the region 150 nt upstream of 3′ splice sites, and the region 150 nt downstream of the 5′ end of the last exon.

FIG. 5D shows an exemplary schematic illustrating a model of NSP9 interacting with nuclear pore complex proteins NUP62, NUP214, NUP58, NUP88 and NUP54, and inhibiting U2AF2 substrate recognition in preventing NXF1 facilitated transport.

FIG. 5E shows cumulative distributive plot (CDF) of log 2(Fold Change) of BEAS-2B cells overexpressing NSP9 versus wildtype BEAS-2B cells in each of nuclear, cytosolic, and total mRNA fractions. Solid line indicate NSP9 target genes, dashed lines indicate genes that are not NSP9 targets.

FIG. 5F shows genome browser tracks of NSP9 eCLIP target RNA mapped to IL-1α, IL-1β, ANXA2 and UPP1.

FIG. 5G shows a bar plot showing ratios of cytosolic to total fraction of mRNA levels measured by RTqPCR, in wild type (WT) BEAS-2B cells, and BEAS-2B cells transduced to express NSP9 (*p<0.05, **p<0.0005, two-tailed multiple t-test with pooled variance, n=2 biologically independent replicates).

FIG. 5H shows a bar plot showing mean concentration of IL-1α in culture media from WT and NSP9 expressing BEAS-2B cells, 48h after induction by cytokines indicated on the x-axis (US, unstimulated; mean±s.e.m, n=3 biologically independent replicates; *p<0.05, Tukey's multiple comparisons test).

FIG. 5I shows a bar plot showing mean concentration of IL-1α in culture media from WT and NSP9 expressing BEAS-2B cells, 48h after induction by different levels of TNFα (mean±s.e.m, n=3 biologically independent replicates, *p<0.05, **p<0.005, two-tailed t-test).

FIG. 5J shows a bar plot showing mean concentration of IL-10 in culture media from WT and NSP9 expressing BEAS-2B cells, 48h after induction by 0 or 100 ng/ml TNFα (mean±s.e.m, n=3 biologically independent replicates, *p<0.05, **p<0.005, two-tailed t-test).

FIG. 6A shows an exemplary schematic illustrating the complex host-viral relationship. Flat-ended arrows indicate downregulation, pointed arrows indicate upregulation. Blue arrows are newly proposed interactions.

FIG. 6B shows an exemplary schematic illustrating the hypothesis that siRNA knockdown of host genes upregulated by SARS-CoV-2 proteins would result in reduced virus proliferation if the genes are pro-viral.

FIG. 6C shows an exemplary schematic outlining the workflow of testing the antiviral effect of siRNAs targeting candidate pro-viral genes. siRNAs are transfected at 25 nM. Cells are infected at MOI of 3 for A549-ACE2 cells and MOI of 1 for lung organoid cells.

FIGS. 6D-6E show bar plots showing the infection rate (mean fluorescence intensity of nucleocapsid divided by cell count) by normalized to the scrambled siRNA control in A549-ACE2 cells (FIG. 6D) and lung organoid cells (FIG. 6E). Cells were treated with siRNAs that target host genes, as indicated in the x-axis (mean±s.e.m, n=3 biologically independent replicates; * p<0.05, ** p<0.005, two-tailed Welch's t-test; ns, not significant).

FIG. 7A shows secondary structures that overlap with the major peaks enriched in NSP12 eCLIP at positions 3533-3635, 17202-17222, 21177-21206, and 24018-24079.

FIG. 7B shows results from a filter-binding assay showing RNA crosslinked to NSP12 on the nitrocellulose layer, and the free RNA on the nylon layer.

FIG. 7C shows multiple sequence alignment of the region corresponding among the SARS-CoV-2 reference sequence, and homologous bat and pangolin sequences. Consensus sequence and structure are indicated underneath the aligned sequences.

FIG. 7D shows a phylogenetic tree constructed from FIG. 7C.

FIG. 7E shows a RNA-seq read density plot from SARS-CoV-2 infected A549-ACE2 cells mapping sequenced reads to the sense strand of the SARS-CoV-2 genome.

### DETAILED DESCRIPTION

Detailed herein are methods of modulating gene expression of a target RNA that include delivering an exogenous nucleic acid into a cell, wherein the exogenous nucleic acid comprises an antisense oligonucleotide or a small interfering RNA (siRNA), and wherein the exogenous nucleic acid binds to the target RNA, thereby modulating gene expression of the target RNA in the cell.

Various non-limiting aspects of these methods are described herein, and can be used in any combination without limitation. Additional aspects of various components of methods for modulating gene expression are known in the art.

It must be noted that, as used in the specification and the appended claims, the singular forms “a,” “an” and “the” include plural referents unless the context clearly dictates otherwise.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

Unless otherwise specified, a “nucleotide sequence encoding a protein” includes all nucleotide sequences that are degenerate versions of each other and thus encode the same amino acid sequence.

As used herein, the term “plurality” can refer to a state of having a plural (e.g., more than one) number of different types of things (e.g., a cell, a genomic sequence, a subject, a system, or a protein). In some embodiments, a plurality of genomic sequences can be more than one genomic sequence wherein each genomic sequence is different from each other.

As used herein, the term “subject” is intended to include any mammal. In some embodiments, the subject is cat, a dog, a goat, a human, a non-human primate, a rodent (e.g., a mouse or a rat), a pig, or a sheep.

As used herein, the term “transduced”, “transfected”, or “transformed” refers to a process by which exogenous nucleic acid is introduced or transferred into a cell. A “transduced,” “transfected,” or “transformed” mammalian cell is one that has been transduced, transfected or transformed with exogenous nucleic acid (e.g., a gene delivery vector) that includes an exogenous nucleic acid encoding RNA-binding zinc finger domain).

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

### Inhibitory Nucleic Acids

Disclosed herein are inhibitory nucleic acids which block the interaction between a host and SARS-CoV2-associated RNA or protein. In some embodiments, the inhibitory nucleic acids are oligonucleotides. In some embodiments, the inhibitory nucleic acids are antisense oligonucleotides. In some embodiments, the inhibitory nucleic acids are modified oligonucleotides. In some embodiments, the inhibitory nucleic acids are locked nucleic acids (LNAs). In some embodiments, the modified oligonucleotides are antisense oligonucleotides that hybridize to viral RNA. In some embodiments, the inhibitory nucleic acids are designed to target a specific region of viral RNA. For example, a specific functional region can be targeted. In some embodiments, the inhibitory nucleic acids hybridize to a viral genome. In some embodiments, the inhibitory nucleic acids are complementary to and/or hybridize to SARS-CoV2. In some embodiments, the inhibitory nucleic acids can be used in model systems, e.g., lung organoids. In some embodiments, the inhibitory nucleic acids are complementary to and/or hybridize and/or bind to host RNA. In some embodiments, the inhibitory nucleic acids hybridize to the polyA tail of host mRNA.

In some embodiments, the inhibitory nucleic acids can be used to treat viral infections in a host. In some embodiments, the inhibitory nucleic acids can be used in pharmaceutical compositions to achieve a therapeutic effect, e.g., reducing or alleviating the symptoms of a disease in a patient. In some embodiments, the disease is a viral infection. In some embodiments, the therapeutic effect includes a reduction or elimination of symptoms including fever or chills, cough, shortness of breath or difficulty breathing, fatigue, muscle or body aches, headache, new loss of taste or smell, sore throat, congesting or runny nose, nausea or vomiting, diarrhea.

In some embodiments, the pharmaceutical composition comprises an inhibitory nucleic acid, e.g., an antisense oligonucleotide that is complementary to one or more viral RNAs, which may cause the disease in the patient. In some embodiments, the inhibitory nucleic acid may be complementary to one or more host RNAs. In some embodiments, as described in further detail below, the antisense oligonucleotide includes different modifications, e.g., in the sugar backbone to make it more cell permeable and nuclease resistant and physiologically non-toxic at low concentrations.

Inhibitory nucleic acids useful in the present methods and compositions include antisense oligonucleotides, modified bases/locked nucleic acids (LNAs), antagomirs, peptide nucleic acids (PNAs), double stranded RNA species, siRNAs, morpholinos, and other oligomeric compounds or oligonucleotide mimetics which hybridize to at least a portion of the target nucleic acid (i.e., an viral RNA gene, e.g., a SARS-CoV2 gene) and modulate its abundance, splicing, post-transcriptional processing, or translation; see, e.g., U.S. Pat. Nos. 9,045,749 and 9,476,046. In some embodiments, the inhibitory nucleic acids include antisense RNA, antisense DNA, chimeric antisense oligonucleotides, antisense oligonucleotides comprising modified linkages, or combinations thereof. See, e.g., WO 2010040112.

In some embodiments, the inhibitory nucleic acids are LNAs. A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

In some embodiments, LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a SARS-CoV2 sequence. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA (or any other inhibitory nucleic acid described herein); for example, a series of oligonucleotides of 10-30 nucleotides spanning the length of a target SARS-CoV2 sequence can be prepared, followed by testing for activity. Optionally, gaps, e.g., of 5-10 nucleotides or more, can be left between the LNAs to reduce the number of oligonucleotides synthesized and tested. GC content is preferably between about 30 and 60%. General guidelines for designing LNAs are known in the art; for example, LNA sequences will bind very tightly to other LNA sequences, so it is preferable to avoid significant complementarity within an LNA. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

In some embodiments, the inhibitory nucleic acids are siRNA. The term “siRNA” refers to small inhibitory RNA duplexes that induce the RNA interference (RNAi) pathway. As used herein, a “small interfering RNA” (siRNA) sometimes known as “short interfering RNA” or “silencing RNA”, refers to a class of double-stranded RNA (e.g., non-coding RNA molecules), typically 18-30 base pairs, 20-24 base pairs, (e.g., 20 base pairs, 21 base pairs, 22 base pairs, 23 base pairs, or 24 base pairs) in length, and operating within the RNA interference (RNAi) pathway.

RNA interference can offer a therapeutic approach for treating viral infections, wherein small interfering RNAs (siRNAs) can be used to target a viral sequence and inhibit (i.e., reduce or eliminate) viral gene expression, and also for treatment of a viral infection in a mammal, such as a human. In some embodiments, the small interfering RNA constructs can inhibit gene expression of a virus by inducing cleavage of viral polynucleotide sequences within or near the target RNA sequence that is recognized by the antisense sequence of the small interfering RNA. In some embodiments, siRNAs interfere with the expression of specific genes with complementary nucleotide sequences by degrading mRNA after transcription, preventing translation. These small inhibitory RNA molecules can vary in length (generally 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region.

In some embodiments, a small interfering RNA can refer to an RNA construct that contains one or more short sequences that are at least partially complementary to and can interact with a polynucleotide sequence of a virus (e.g., viral RNA). Interaction may be in the form of a direct binding between complementary (antisense) sequences of the small interfering RNA and polynucleotide sequences of the viral target, or in the form of an indirect interaction via enzymatic machinery (e.g., a protein complex) that allows the antisense sequence of the small interfering RNA to recognize the target sequence. In some embodiments, recognition of the target sequence by the small interfering RNA results in cleavage of viral sequences within or near the target site that is recognized by the recognition (antisense) sequence of the small interfering RNA. The small interfering RNA can exclusively contain ribonucleotide residues, or the small interfering RNA can contain one or more modified residues, particularly at the ends of the small interfering RNA or on the sense strand of the small interfering RNA. In some embodiments, small interfering RNA encompasses shRNA and siRNA, both of which are understood and known to those in the art to refer to RNA constructs with particular characteristics and types of configurations.

In some embodiments, the inhibitory nucleic acids are antisense oligonucleotides. Antisense oligonucleotides are typically designed to inhibit expression of a DNA or RNA target by binding to the target and halting expression at the level of transcription, translation, or splicing. Antisense oligonucleotides of the present compositions and methods are complementary nucleic acid sequences designed to a target viral RNA to achieve a therapeutic effect in a patient. Thus, inhibitory nucleic acids and/or oligonucleotides are chosen that are sufficiently complementary to the target, i.e., that hybridize sufficiently well and with sufficient specificity, to give the desired therapeutic effect.

In some embodiments, the inhibitory nucleic acids are 9 to 50, 9 to 21, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therein. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies inhibitory nucleic acids having antisense portions of 12, 13, 14, 15, 16, 17, 18, 19, 20, 21, 22, 23, 24, 25, 26, 27, 28, 29 or 30 nucleotides in length, or any range therein.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; Fluiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Orom et al., Gene. 2006 May 10; 372( ):137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ (known as a methylene(methylimino) or MMI backbone], CH2-O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH₂)nCH₃, O(CH₂)nNH₂ or O(CH₂)nCH₃ where n is from 1 to about 10; Ci to C10 lower alkyl, alkoxyalkoxy, substituted lower alkyl, alkaryl or aralkyl; Cl; Br; CN; CF₃; OCF₃; O-, S, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)](Martin et al, HeIv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A“universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, a palmityl moiety, or an octadecylamine or hexylamino-carbonyl-t oxycholesterol moiety.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention.

The inhibitory nucleic acids useful in the present compositions and methods are sufficiently complementary to all or part of a target viral RNA, i.e., hybridize sufficiently well and with sufficient specificity, to give the desired therapeutic effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a target viral RNA, then the bases are considered to be complementary to each other at that position. 100% complementarity is not required.

In the context of the present compositions and methods, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. The inhibitory nucleic acids and the target viral RNA are complementary to each other when a sufficient number of corresponding positions in each molecule are occupied by nucleotides that can hydrogen bond with each other. Thus, “specifically hybridizable” and “complementary” are terms which are used to indicate a sufficient degree of complementarity or precise pairing such that stable and specific binding occurs between the inhibitory nucleic acid and the target viral RNA. For example, if a base at one position of an inhibitory nucleic acid is capable of hydrogen bonding with a base at the corresponding position of the target viral RNA, then the bases are considered to be complementary to each other at that position.

Although in some embodiments, 100% complementarity is desirable, it is understood in the art that a complementary nucleic acid sequence need not be 100% complementary to that of its target nucleic acid to be specifically hybridisable. A complementary nucleic acid sequence for purposes of the present compositions and methods is specifically hybridisable when binding of the sequence to the target viral RNA interferes with the abundance, splicing, post-transcriptional processing, or translation of the mRNA transcript, and there is a sufficient degree of complementarity to avoid non-specific binding of the sequence to non-target nucleic acid molecules under conditions in which specific binding is desired, e.g., under physiological conditions.

In general, the inhibitory nucleic acids useful in the compositions and methods described herein have at least 80% sequence complementarity to a target region within the target nucleic acid, e.g., 90%, 91%, 92%, 93%, 94%, 95%, 96%, 97%, 98%, 99% or 100% sequence complementarity to the target region. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). In general the inhibitory nucleic acids must retain specificity for their target, i.e., must not directly bind to, or directly significantly affect expression levels of, transcripts other than the intended target viral RNA.

In some embodiments, the inhibitory nucleic acid is capable of reducing viral infection in a human cell (e.g., HEK293 cell, an iPSC cell, BEAS-2B cell, or A549-ACE2 cell). In some embodiments, the viral infection is reduced by at least about 30%, by at least about 35%, by at least about 40%, by at least about 45%, by at least about 50%, by at least about 55%, by at least about 60%, by at least about 65%, by at least about 70%, by at least about 75%, by at least about 80%, by at least about 85%, by at least about 90%, by at least about 95%, or about 100% compared to a human cell that is not exposed to the inhibitory nucleotide.

In some embodiments, the pharmaceutical composition is an inhibitory nucleic acids comprising a sequence selected from SEQ ID NOs: 1-40 (Table 1). In some embodiments, the inhibitory nucleic acid includes a nucleic acid complementary to a sequence selected from SEQ ID NOs: 1-40. In some embodiments, the inhibitory nucleic acid includes a double stranded inhibitory nucleic acid comprising at least one sequence selected from SEQ ID NO: 140. In some embodiments, an inhibitory nucleic acid includes a sequence complement to at least one sequence selected from SEQ ID NO: 1-40. In some embodiments, the pharmaceutical composition is an inhibitory nucleotide comprising any of SEQ ID NOs: 1-40 (and/or its complement) wherein all or some of the nucleotides have been modified in one or more ways as described above, e.g., with a phosphorothioate backbone, one or more 2′-methoxyethoxy modifications, one or more 2′-methoxy modifications, one or more 2′-propoxy modifications, or one or more 2′-fluoro modifications.

In some embodiments, the pharmaceutical composition comprises any of SEQ ID NOs: 1-40, and/or its complement. In some embodiments the pharmaceutical composition comprising any of SEQ ID NOs: 1-40, and its complement, is therapeutically effective for treating a disease caused by SARS-CoV2.

TABLE 1

| siRNA | | | SEQ |
|---|---|---|---|
| target | | | ID |
| gene | DsiRNA name | Sequence 5′→3′ (+strand, −strand)# | NO |
| HSPA5 | hs.Ri.HSPA5.13.1 | rUrCrUrArCrArGrCrUrUrCrUrGrArUrArArUrCrArArCrCAA, | 1 |
| | | rUrUrGrGrUrUrGrArUrUrArUrCrArGrArArGrCrUrGrUrArGrArArA | 2 |
| RHOA | hs.Ri.RHOA.13.1 | rCrUrUrGrCrUrArCrCrArGrUrArUrUrUrArGrArArGrCrCAA, | 3 |
| | | rUrUrGrGrCrUrUrCrUrArArArUrArCrUrGrGrUrArGrCrArArGrArU | 4 |
| PGK1 | hs.Ri.PGK1.13.1 | rArCrCrUrUrCrCrArUrGrUrCrArArGrArUrUrCrArGrCrUAG, | 5 |
| | | rCrUrArGrCrUrGrArArUrCrUrUrGrArCrArUrGrGrArArGrGrUrUrU | 6 |
| NDUFA4 | hs.Ri.NDUFA4.13.1 | rArArArUrCrArUrGrUrUrGrGrArGrArUrCrUrCrUrArUrUGT, | 7 |
| | | rArCrArArUrArGrArGrArUrCrUrCrCrArArCrArUrGrArUrUrUrCrA | 8 |
| LAMP1 | hs.Ri.LAMP1.13.1 | rArArGrGrArArUrCrCrArGrUrUrGrArArUrArCrArArUrUCT, | 9 |
| | | rArGrArArUrUrGrUrArUrUrCrArArCrUrGrGrArUrUrCrCrUrUrGrU | 10 |
| LAPTM4A | hs.Ri.LAPTM4A.13.1 | rArGrUrArUrGrArArGrUrCrArUrCrGrGrUrArArUrUrArCTA, | 11 |
| | | rUrArGrUrArArUrUrArCrCrGrArUrGrArCrUrUrCrArUrArCrUrGrA | 12 |
| PSMD13 | hs.Ri.PSMD13.13.3 | rCrGrGrUrUrUrGrArGrArArUrGrUrUrCrCrUrArUrArArUAA, | 13 |
| | | rUrUrArUrUrArUrArGrGrArArCrArUrUrCrUrCrArArArCrCrGrCrA | 14 |
| LDHB | hs.Ri.LDHB.13.3 | rGrArGrCrCrUrUrUrArGrUrUrUrUrCrArUrCrCrArUrGrUAC, | 15 |
| | | rGrUrArCrArUrGrGrArUrGrArArArArCrUrArArArGrGrCrUrCrGrA | 16 |
| UGGT1 | hs.Ri.UGGT1.13 | rArGrCrUrGrArGrArUrGrUrUrCrCrUrUrArGrUrArArUrCAT, | 17 |
| | TriFECTa Kit | rArUrGrArUrUrArCrUrArArGrGrArArCrArUrCrUrCrArGrCrUrGrU; | 18 |
| | DsiRNA | rGrGrCrUrCrArGrCrUrGrArUrArArArCrArUrGrArArUrCTG, | 19 |
| | | rCrArGrArUrUrCrArUrGrUrUrUrArUrCrArGrCrUrGrArGrCrCrArA; | 20 |
| | | rGrArArUrGrGrArArArUrGrUrArUrUrGrGrUrArArArGrCTA, | 21 |
| | | rUrArGrCrUrUrUrArCrCrArArUrArCrArUrUrUrCrCrArUrUrCrCrC | 22 |
| C1QBP | hs.Ri.C1QBP.13.1 | rArGrUrCrUrGrArArUrGrGrArArGrGrArUrArCrUrArArUTA, | 23 |
| | | rUrArArUrUrArGrUrArUrCrCrUrUrCrCrArUrUrCrArGrArCrUrCrG | 24 |
| RPN1 | hs.Ri.RPN1.13.1 | rGrUrUrCrUrGrArArGrUrCrUrArArGrArUrArUrUrUrUrUCA, | 25 |
| | | rUrGrArArArArArUrArUrCrUrUrArGrArCrUrUrCrArGrArArCrArG | 26 |
| OST4 | hs.Ri.OST4.13.1 | rArArCrUrArCrArCrCrUrGrGrArUrUrUrUrUrCrCrArArACA, | 27 |
| | | rUrGrUrUrUrGrGrArArArArArUrCrCrArGrGrUrGrUrArGrUrUrGrC | 28 |
| ACE2 | hs.Ri.ACE2.13.1 | rArGrUrGrArUrGrUrUrUrGrGrArArUrCrGrArUrCrArUrGCT, | 29 |
| | | rUrGrUrCrArCrUrArCrArArArCrCrUrUrArGrCrUrArGrUrArCrGrA | 30 |
| PRSS23 | hs.Ri.PRSS23.13.1 | rGrArGrCrUrUrUrUrUrGrGrArArGrGrArUrArArUrUrCrUGA, | 31 |
| | | rUrCrArGrArArUrUrArUrCrCrUrUrCrCrArArArArArGrCrUrCrArA | 32 |
| HSP90AB1 | hs.Ri.HSP90AB1.13.1 | rGrGrArCrArGrUrGrGrUrArArArGrArGrCrUrGrArArArATT, | 33 |
| | | rArArUrUrUrUrCrArGrCrUrCrUrUrUrArCrCrArCrUrGrUrCrCrArA | 34 |
| ATP5B | hs.Ri.ATP5B.13.1 | rGrGrUrCrArArGrArUrGrUrArCrUrGrCrUrArUrUrUrArUTG, | 35 |
| | | rCrArArUrArArArUrArGrCrArGrUrArCrArUrCrUrUrGrArCrCrUrU | 36 |
| Scrambled | Scrambled Negative | /5Phos/rCrUrUrCrCrUrCrUrCrUrUrUrCrUrCrUrCrCrCrUrUrGrUGA, | 37 |
| | Control DsiRNA | rUrCrArCrArArGrGrGrArGrArGrArArArGrArGrArGrGrArArGrGrA | 38 |
| Transfection | TYE 563 | /5TYE563/T*CrCrUrUrCrCrUrCrUrCrUrUrUrCrUrCrUrCrCrCrUrUr | 39 |
| Control | Transfection Control | GrUG*A, | |
| | DsiRNA | /5TYE563/T*CrArCrArArGrGrGrArGrArGrArArArGrArGrArGrGrAr | 40 |
| | | ArGG*A | |

#where rN is a ribonucleoside for rN = {rA, rU, rC, rG}, and N without any r prepended is a deoxyribonucleoside for N = {A, T, C, G}

### Pharmaceutical Compositions

The methods described herein can include the administration of pharmaceutical compositions and formulations comprising inhibitory nucleic acid sequences designed to target SARS-CoV2 RNA or host RNA that interacts with SARS-CoV2 proteins.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. The compositions may conveniently be presented in unit dosage form and may be prepared by any methods well known in the art of pharmacy. The amount of active ingredient (e.g., nucleic acid sequences of this invention) which can be combined with a carrier material to produce a single dosage form can vary depending upon the host being treated, the particular mode of administration. The amount of active ingredient which can be combined with a carrier material to produce a single dosage form will generally be that amount of the compound which produces a therapeutic effect, e.g., reduction in SARS-CoV2 infection levels and/or reduction in a symptom of COVID19.

Pharmaceutical compositions described herein can be prepared according to any method known to the art for the manufacture of pharmaceuticals. Such compositions can contain, for example, preserving agents. A composition can be admixtured with nontoxic pharmaceutically acceptable excipients which are suitable for manufacture. Compositions may comprise one or more diluents, emulsifiers, preservatives, buffers, excipients, etc. and may be provided in such forms as liquids, powders, emulsions, lyophilized powders, controlled release formulations, on patches, in implants, etc. Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences as described herein. As an example of an injectable oil vehicle, see Minto (1997) J. Pharmacol. Exp. Ther. 281:93-102.

Pharmaceutical compositions can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

In some embodiments, the pharmaceutical compositions can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compositions can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

Compositions disclosed herein can be administered for prophylactic and/or therapeutic treatments. In some embodiments, for therapeutic applications, compositions are administered to a subject who is infected or at risk of infection with SARS-CoV2, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease the number of lung cells infected with SARS-CoV2.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age, and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regimen, i.e., dose schedule and dosage levels, administered practicing the methods of the disclosure are correct and appropriate.

Single or multiple administrations of compositions can be given depending on for example: the dosage and frequency as required and tolerated by the patient, the degree and amount of therapeutic effect generated after each administration (e.g., effect on symptoms), and the like. The compositions should provide a sufficient quantity of active agent to effectively treat, prevent or ameliorate conditions, diseases or symptoms.

The inhibitory nucleic acids used to practice the methods described herein, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect, or plant cell expression systems.

Inhibitory nucleic acids of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus, or alphavirus. The recombinant vectors capable of expressing the nucleic acids of the invention can be delivered as described herein, and persist in target cells (e.g., stable transformants).

### Methods of Treating Coronavirus Disease 2019 (COVID-19)

Coronavirus disease (COVID-19) is caused by the novel coronavirus SARS-CoV-2. The novel severe acute respiratory syndrome coronavirus 2 (SARS-CoV-2, and/or any variant of SARS-CoV2) is a positive-sense single-stranded (+ss) RNA virus, wherein the viral genome encodes 29 proteins, which include the four structural proteins, membrane or matrix (M), nucleocapsid (N), envelope (E), and spike (S) proteins. In addition, there are 16 non-structural proteins, NSP1-16, and 9 accessory proteins ORF3a-ORF10, though the expression of some of the accessory factors are still debated.

Provided herein are methods of treating Coronavirus disease 2019 (COVID-19) in a subject that include administering to the subject a therapeutically effective amount of a composition comprising an exogenous nucleic acid (e.g., an inhibitory nucleic acid) and delivering the exogenous nucleic acid into a cell, wherein the exogenous nucleic acid binds to a target RNA and modulates gene expression of the target RNA, thereby treating Coronavirus disease 2019 (COVID-19) in the subject.

As used herein, the term “exogenous nucleic acid” refers to any DNA or RNA, or a combination thereof, in either a single- or double-stranded form that is introduced from or originating from outside a cell, a tissue or an organism that is not produced by or does not originate from the same cell, tissue, or organism in which it is being introduced. In some embodiments, the exogenous nucleic acid is a single stranded nucleic acid. In some embodiments, the exogenous nucleic acid is a double stranded nucleic acid. In some embodiments the exogenous nucleic acid is an inhibitory nucleic acid. In some embodiments, the exogenous nucleic acid (e.g., inhibitory nucleic acid) is one of the inhibitory nucleic acids described herein.

In some embodiments, the inhibitory nucleotides disclosed herein modulate gene expression within a cell. As used herein, “modulating” can refer to modifying, regulating, or altering the endogenous gene expression in a cell. In some embodiments, modulating gene expression can include systematically influencing RNA stability and/or translation by activating or suppressing the gene expression. In some embodiments, modulation of gene expression can include stabilizing a target RNA. In some embodiments, stabilizing a target RNA can increase translation of the target RNA. In some embodiments, modulation of gene expression can include destabilizing a target RNA. In some embodiments, destabilizing a target RNA can suppress translation of the target RNA. In some embodiments, modulation of gene expression can include increasing translation of a target RNA. In some embodiments, modulation of gene expression can include suppressing translation of a target RNA. In some embodiments, the gene expression of the target RNA is upregulated. In some embodiments, the gene expression of the target RNA is downregulated. In some embodiments, the modulating gene expression comprises knockdown of a gene expressed from the target RNA.

In some embodiments, the target RNA interacts with a viral protein. In some embodiments, the target RNA interacting with the viral protein increases protein expression from the gene expressed from the target RNA. In some embodiments, the target RNA interacting with the viral protein inhibits protein expression from the gene expressed from the target RNA.

In some embodiments, the modulating comprises blocking the target RNA from interacting with the viral protein. In some embodiments, the modulating comprises blocking the target RNA from interacting with the viral protein wherein the exogenous nucleic acid binds to the target RNA. In some embodiments, the modulating comprises blocking the target RNA from interacting with the viral protein wherein the exogenous nucleic acid binds to the target RNA by targeting the structured genomic RNA that interacts with the viral protein.

In some embodiments, an endogenous gene is expressed from the target RNA. In some embodiments, the gene expressed from the target RNA is selected from the group consisting of RPN1, UGGT1, OST4, NDUFA4, HSPA5, PSMD13, LAPTMFA, LAMP1, C1QBP, and LDHB.

In some embodiments, the viral protein is a SARS-CoV-2 protein (e.g., SARS-CoV2 and any variant thereof). In some embodiments, the SARS-CoV-2 protein binds to the 5′ UTR, the coding sequence, or the 3′ UTR region of the target RNA. In some embodiments, the SARS-CoV-2 protein binds to the 5′ UTR region of the target RNA. In some embodiments, the SARS-CoV-2 protein binds to the coding sequence region of the target RNA. In some embodiments, the SARS-CoV-2 protein binds to the 3′ UTR region of the target RNA.

In some embodiments, the delivering of an exogenous nucleic acid into a cell comprises transfection of the exogenous nucleic acid into the cell. In some embodiments, the delivering comprises a virus-based delivery of the exogenous nucleic acid into the cell. In some embodiments, the virus-based delivery comprises a lentivirus.

### Coronavirus-Derived Protein

As used herein, the term “viral protein” refers to a protein generated by a virus. A viral protein can be both a component and a product of a virus. Viral proteins can be grouped according to their functions, wherein groups of the viral proteins can include structural proteins, nonstructural proteins, regulatory proteins, and accessory proteins.

Coronaviruses contain a positive-sense, single-stranded RNA genome, and the viral genome consists of more than 29,000 bases and encodes 29 proteins. SARS-CoV-2 has four structural proteins: the E and M proteins, which form the viral envelope; the N protein, which binds to the virus's RNA genome; and the S protein, which binds to human receptors. As used herein, a “coronavirus-derived protein” or “SARS-CoV-2 protein” can refer to a protein that is encoded from the coronavirus viral genome. In some embodiments, the SARS-CoV-2 protein can be a structural protein. In some embodiments, the SARS-CoV-2 protein can be a non-structural protein (NSP). In some embodiments, the SARS-CoV-2 protein can comprise a NSP1, a NSP2, a NSP3, a NSP4, a NSP5, a NSP6, a NSP7, a NSP8, a NSP9, a NSP10, a NSP11, a NSP12, a NSP13, a NSP14, a NSP15, or a NSP16 protein. In some embodiments, the SARS-CoV-2 protein can be an accessory protein. In some embodiments, the SARS-CoV-2 protein can comprise a ORF3a, a ORF3b, a ORF6, a ORF7a, a ORF7b, a ORF8, a ORF9b, a ORF9c, or a ORF10 protein. In some embodiments, the SARS-CoV-2 protein comprises a NSP1, a NSP2, a NSP3, a NSP4, a NSP5, a NSP6, a NSP7, a NSP8, a NSP9, a NSP10, a NSP11, a NSP12, a NSP13, a NSP14, a NSP15, a NSP16, a ORF3a, a ORF3b, a ORF6, a ORF7a, a ORF7b, a ORF8, a ORF9b, a ORF9c, or a ORF10 protein. In some embodiments, the SARS-CoV-2 protein is selected from the group consisting of NSP2, NSP3, NSP6, NSP12, NSP14, ORF3b, ORF7b, and ORF9c.

In some embodiments, NSP2, NSP3, NSP6, NSP12, NSP14, ORF3b, ORF7b, and ORF9c interact with host RNA. In some embodiments, inhibitory nucleic acids block the interaction between a coronavirus-derived protein and a host RNA. In some embodiments, the inhibitory nucleic acid modulates gene expression of a host RNA.

### EXAMPLES

### Example 1—eCLIP Elucidates SARS-CoV-2 Protein-Viral RNA Interactions in Virus Infected Cells

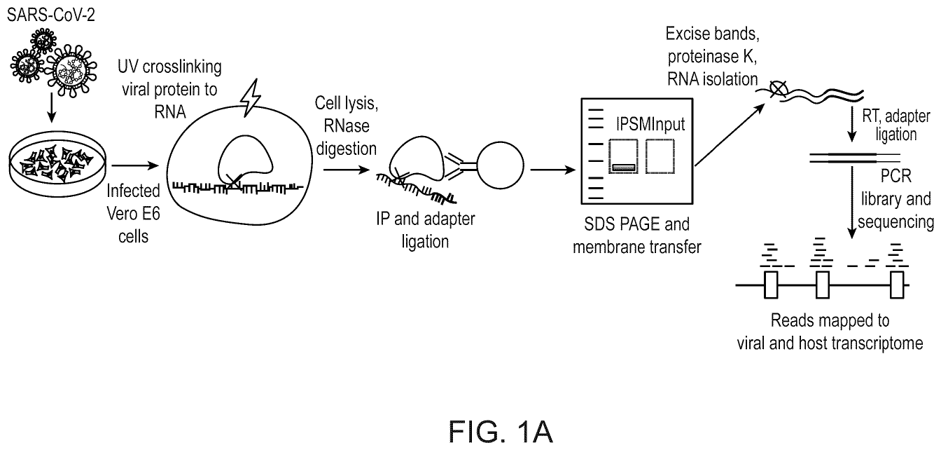

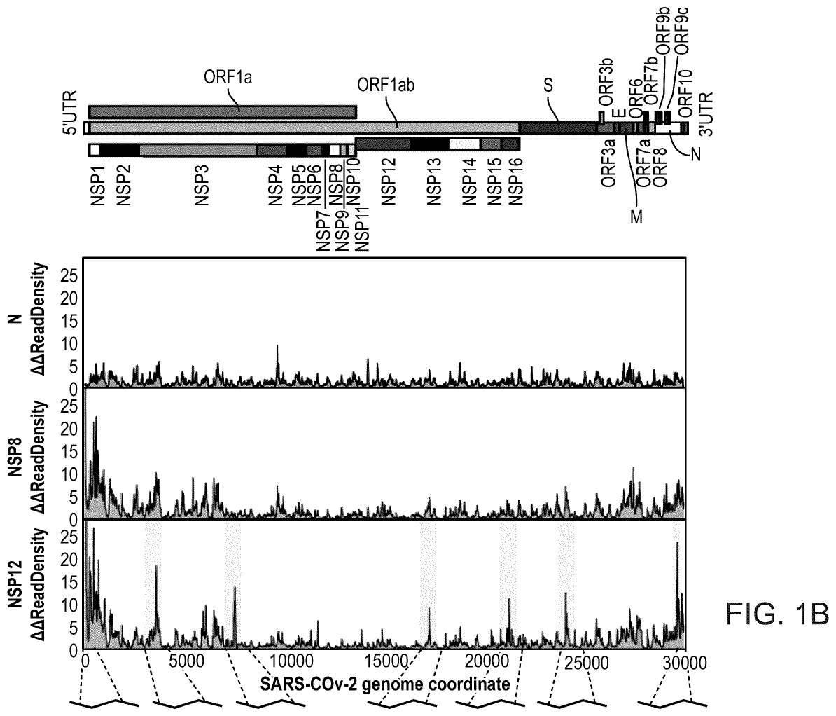

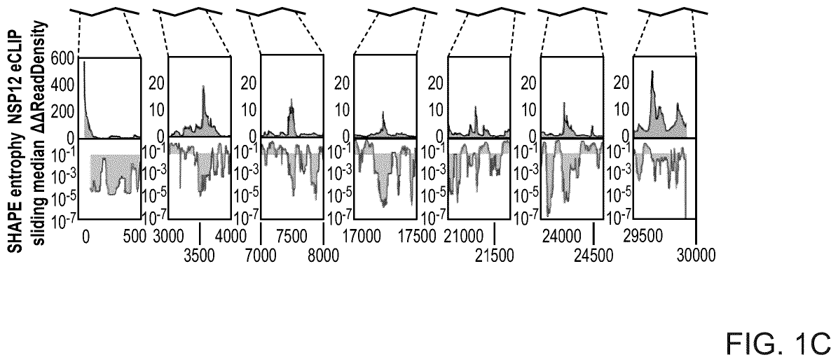

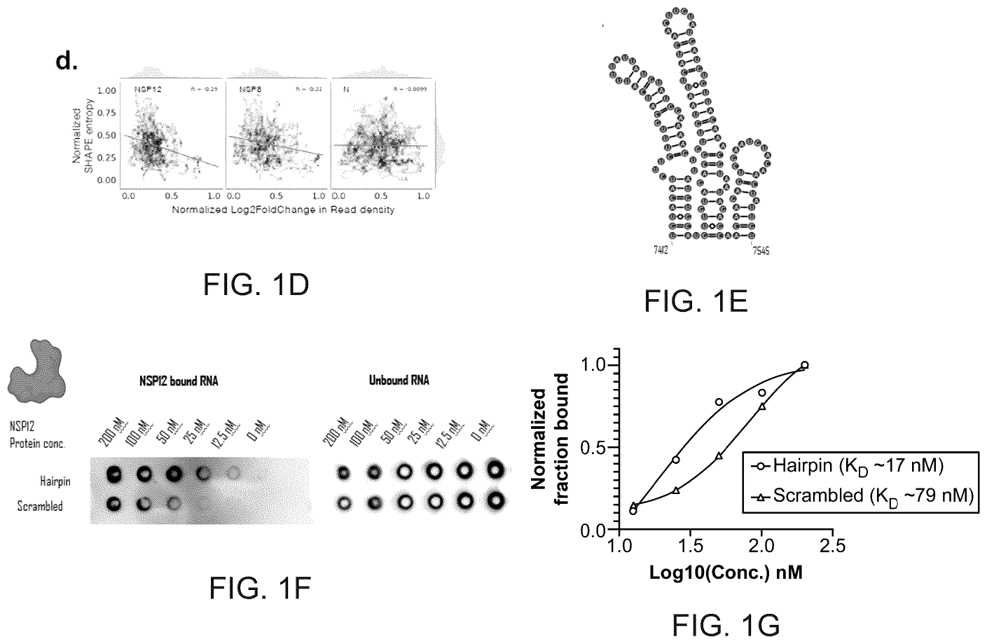

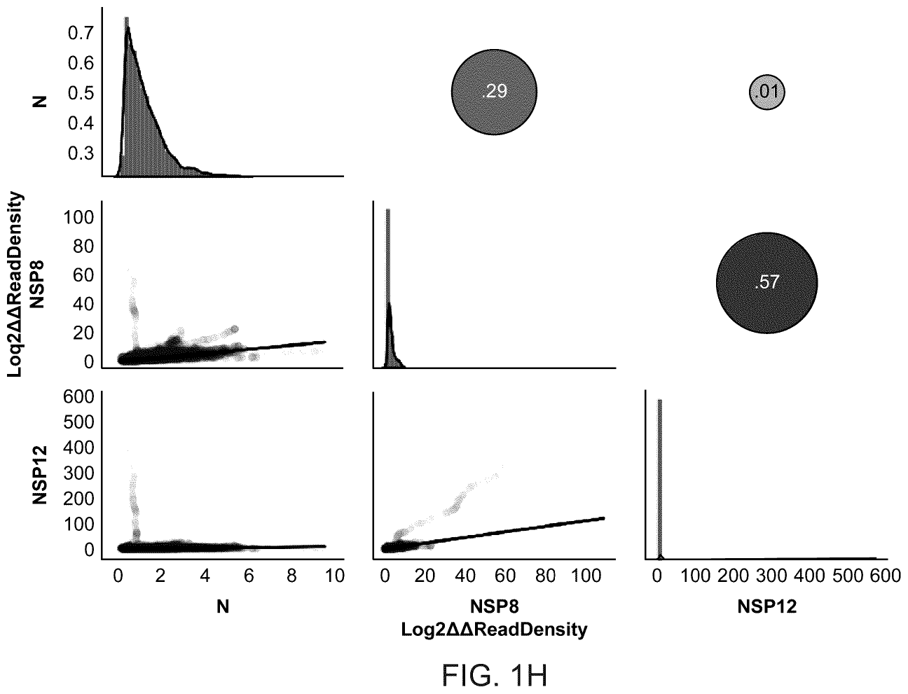

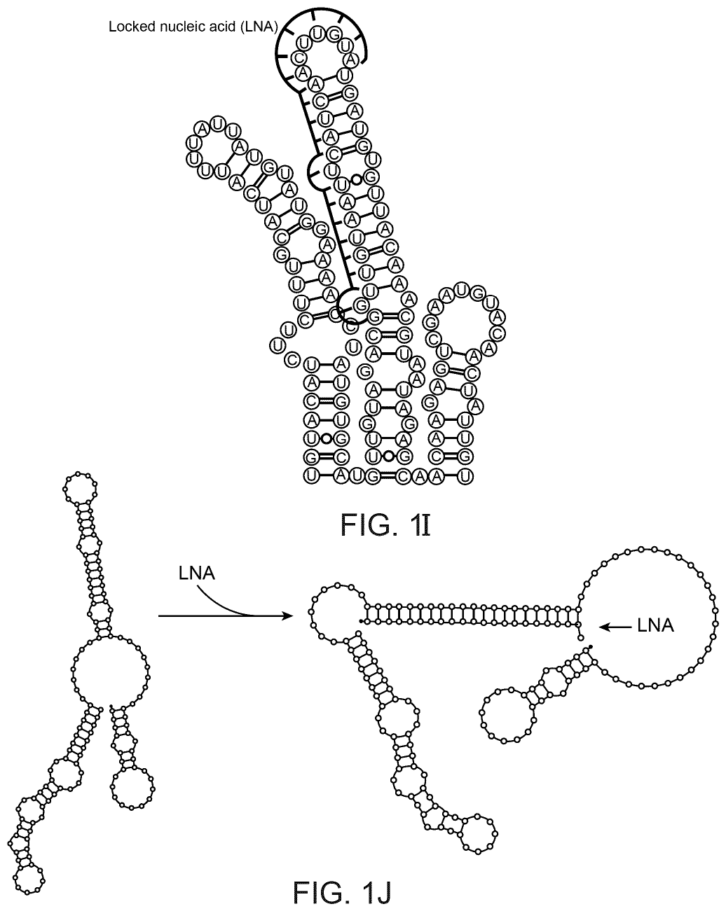

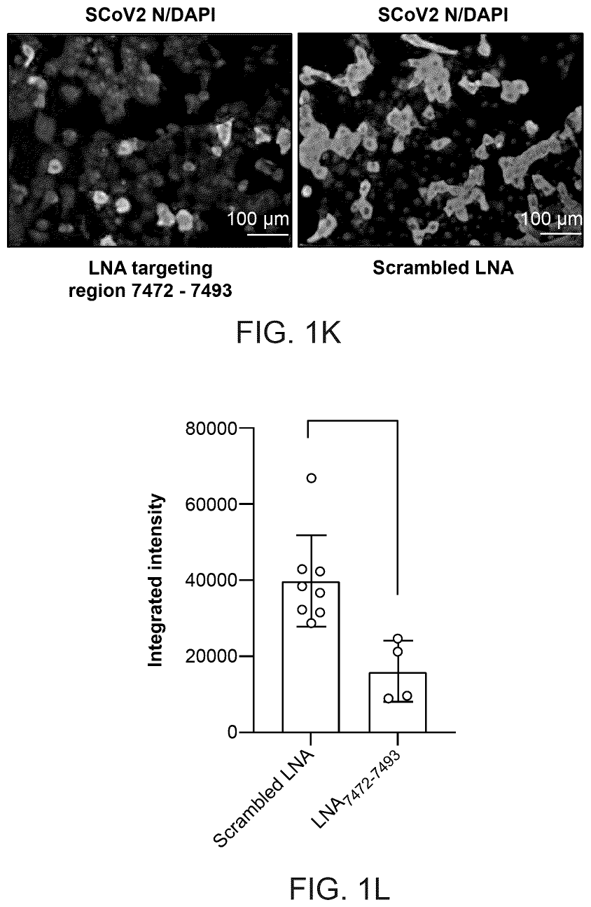

To investigate the RNA interactome of SARS-CoV-2 proteins, eCLIP on SARSCoV-2 infected African Green Monkey kidney (Vero E6) cells was performed, which are an efficiently infected cell line (FIG. 1A). Infected cells were subject to UV irradiation, which covalently crosslinked interacting proteins to RNAs. This was followed by immunoprecipitation of non-structural proteins NSP8 and NSP12, which form part of the replication transcription complex (RTC), and N (nucleocapsid), using protein-specific antibodies to isolate the bound RNA. The RNA-bound proteins were resolved via SDS-PAGE and transferred to nitrocellulose membranes such that only the region spanning the expected protein size and 75 kDa larger were excised and purified in subsequent steps. The same size region of a non-immunoprecipitated input whole cell lysate was included as size-matched input to identify and remove non-specific, enriched sequences. RNA was converted to DNA libraries, sequenced to an average depth of ˜25 million reads, and mapped to the SARS-CoV-2 viral genome and African Green Monkey genome to determine SARS-CoV-2 protein RNA interactions. Thus, reads from the immunoprecipitation (IP) samples correspond to RNA crosslinked to the IP enriched proteins NSP8, NSP12, and N, while reads from the input (IN) samples correspond to RNA crosslinked to RBPs at a similar size to the IP protein in the cell milieu. Normalizing read density of IP to IN samples provide a measure of protein-specific RNA interaction.

The eCLIP results provide a genome-wide map of RNA interactions with viral proteins during an authentic SARS-CoV-2 infection. Reads were mapped across the entire genome in the positive sense for both IP and IN samples for all proteins, with greater than 96% coverage in all input and IP samples. The near complete coverage implies that most of the viral RNA interacts with RNA binding proteins. Two biological replicates were performed for each protein, and read densities show strong replicate agreement in all samples (Pearson's coefficient >0.87). Input-normalized IP read densities also display strong replicate correlation (Pearson's coefficient >0.88).

To identify positions with particularly enriched RBP binding, relative positional enrichment (ΔΔReadDensity) was computed by dividing the fold change of read density of IP over IN at each position by the global median fold change for that sample (FIG. 1B). A strong relative positional enrichment in NSP8 and NSP12 eCLIP read density was observed at the 5′ end, at 573-fold and 103-fold at position 1 respectively, but only 0.75-fold for N. High relative positional enrichment was also observed for the region before position 66, which marks the start of the leader transcription regulatory site (TRS), with >22-fold for NSP12, >4.9-fold for NSP8, and only >0.6-fold for N (Table 2). In both NSP12 and NSP8, there appeared to be a drop in enrichment earlier, around position 33. This corresponds to the end of Stem Loop 1 (SL1) in the 5′ untranslated region (UTR), potentially implicating SL1 in recruiting, stabilizing or otherwise regulating the replicase proteins. At the 3′ end, NSP12 and NSP8 appear strongly enriched (>5-fold relative to global median) after the stop codon in N at position 29,533 up to the start of the S2M structured region in the 3′ UTR at position 29,695. NSP12 and NSP8 continued to be strongly enriched again after the S2M region at position 29,809. The lack of enrichment at the S2M structure may suggest that its function is unrelated to recruiting replicase proteins NSP12 and NSP8. The eCLIP findings thus provide a map of the direct interaction between replicase proteins NSP8 and NSP12 with regions in the UTRs likely involved in regulating replication and transcription.

TABLE 2
SARS-CoV-2 genome regions with >5-fold ΔΔReadDensity

| | Chromosome | Start | End | MaxRel.Enrichment | PeakMaxPosition | PeakLength | Strand | Sample |
|---|---|---|---|---|---|---|---|---|
| 1 | MN908947.3 | 559 | 579 | 5.4 | 567 | 20 | + | N |
| 2 | MN908947.3 | 939 | 961 | 5.6 | 945 | 22 | + | N |
| 3 | MN908947.3 | 3688 | 3713 | 5.9 | 3701 | 25 | + | N |
| 4 | MN908947.3 | 6635 | 6650 | 5.6 | 6640 | 15 | + | N |
| 5 | MN908947.3 | 9602 | 9620 | 9.5 | 9605 | 18 | + | N |
| 6 | MN908947.3 | 14138 | 14158 | 6.4 | 14149 | 20 | + | N |
| 7 | MN908947.3 | 26896 | 26917 | 5.5 | 26902 | 21 | + | N |
| 1 | MN908947.3 | 1 | 133 | 573.2 | 1 | 132 | + | NSP12 |
| 2 | MN908947.3 | 211 | 867 | 27.0 | 391 | 656 | + | NSP12 |
| 3 | MN908947.3 | 932 | 1028 | 11.3 | 983 | 96 | + | NSP12 |
| 4 | MN908947.3 | 1237 | 1369 | 8.8 | 1270 | 132 | + | NSP12 |
| 5 | MN908947.3 | 3533 | 3635 | 18.6 | 3547 | 102 | + | NSP12 |
| 6 | MN908947.3 | 5906 | 5924 | 8.4 | 5908 | 18 | + | NSP12 |
| 7 | MN908947.3 | 6042 | 6067 | 9.7 | 6046 | 25 | + | NSP12 |
| 8 | MN908947.3 | 6427 | 6508 | 8.8 | 6435 | 81 | + | NSP12 |
| 9 | MN908947.3 | 6564 | 6602 | 6.8 | 6570 | 38 | + | NSP12 |
| 10 | MN908947.3 | 7436 | 7526 | 13.7 | 7489 | 90 | + | NSP12 |
| 11 | MN908947.3 | 17202 | 17222 | 9.2 | 17206 | 20 | + | NSP12 |
| 12 | MN908947.3 | 21177 | 21206 | 11.2 | 21181 | 29 | + | NSP12 |
| 13 | MN908947.3 | 24018 | 24079 | 12.5 | 24022 | 61 | + | NSP12 |
| 14 | MN908947.3 | 25541 | 25558 | 6.8 | 25547 | 17 | + | NSP12 |
| 15 | MN908947.3 | 27188 | 27280 | 9.0 | 27202 | 92 | + | NSP12 |
| 16 | MN908947.3 | 27576 | 27629 | 7.5 | 27581 | 53 | + | NSP12 |
| 17 | MN908947.3 | 27645 | 27733 | 6.9 | 27689 | 88 | + | NSP12 |
| 18 | MN908947.3 | 28391 | 28404 | 5.4 | 28398 | 13 | + | NSP12 |
| 19 | MN908947.3 | 28912 | 28933 | 5.8 | 28920 | 21 | + | NSP12 |
| 20 | MN908947.3 | 29035 | 29052 | 5.4 | 29041 | 17 | + | NSP12 |
| 21 | MN908947.3 | 29555 | 29693 | 23.8 | 29579 | 138 | + | NSP12 |
| 22 | MN908947.3 | 29794 | 29880 | 12.4 | 29819 | 86 | + | NSP12 |
| 1 | MN908947.3 | 1 | 68 | 106.8 | 1 | 67 | + | NSP8 |
| 2 | MN908947.3 | 226 | 323 | 12.6 | 273 | 97 | + | NSP8 |
| 3 | MN908947.3 | 332 | 380 | 6.8 | 348 | 48 | + | NSP8 |
| 4 | MN908947.3 | 382 | 513 | 21.3 | 435 | 131 | + | NSP8 |
| 5 | MN908947.3 | 520 | 592 | 22.5 | 565 | 72 | + | NSP8 |
| 6 | MN908947.3 | 603 | 892 | 15.1 | 642 | 289 | + | NSP8 |
| 7 | MN908947.3 | 922 | 1010 | 10.7 | 950 | 88 | + | NSP8 |
| 8 | MN908947.3 | 1215 | 1369 | 8.8 | 1242 | 154 | + | NSP8 |
| 9 | MN908947.3 | 2463 | 2473 | 5.5 | 2470 | 10 | + | NSP8 |
| 10 | MN908947.3 | 2568 | 2628 | 7.4 | 2599 | 60 | + | NSP8 |
| 11 | MN908947.3 | 3452 | 3489 | 7.1 | 3465 | 37 | + | NSP8 |
| 12 | MN908947.3 | 3534 | 3726 | 10.1 | 3548 | 192 | + | NSP8 |
| 13 | MN908947.3 | 4868 | 4886 | 6.2 | 4874 | 18 | + | NSP8 |
| 14 | MN908947.3 | 4932 | 4947 | 5.7 | 4932 | 15 | + | NSP8 |
| 15 | MN908947.3 | 5389 | 5419 | 9.0 | 5398 | 30 | + | NSP8 |
| 16 | MN908947.3 | 6016 | 6095 | 7.8 | 6041 | 79 | + | NSP8 |
| 17 | MN908947.3 | 6425 | 6473 | 8.9 | 6438 | 48 | + | NSP8 |
| 18 | MN908947.3 | 6478 | 6495 | 5.2 | 6482 | 17 | + | NSP8 |
| 19 | MN908947.3 | 6561 | 6602 | 7.4 | 6574 | 41 | + | NSP8 |
| 20 | MN908947.3 | 6626 | 6677 | 7.8 | 6647 | 51 | + | NSP8 |
| 21 | MN908947.3 | 9602 | 9625 | 7.2 | 9605 | 23 | + | NSP8 |
| 22 | MN908947.3 | 9669 | 9683 | 5.6 | 9675 | 14 | + | NSP8 |
| 23 | MN908947.3 | 23574 | 23590 | 5.6 | 23579 | 16 | + | NSP8 |
| 24 | MN908947.3 | 24020 | 24048 | 7.1 | 24026 | 28 | + | NSP8 |
| 25 | MN908947.3 | 24069 | 24079 | 6.0 | 24071 | 10 | + | NSP8 |
| 26 | MN908947.3 | 25551 | 25579 | 5.7 | 25557 | 28 | + | NSP8 |
| 27 | MN908947.3 | 26896 | 26927 | 6.4 | 26902 | 31 | + | NSP8 |
| 28 | MN908947.3 | 27152 | 27162 | 5.4 | 27156 | 10 | + | NSP8 |
| 29 | MN908947.3 | 27170 | 27218 | 6.8 | 27190 | 48 | + | NSP8 |
| 30 | MN908947.3 | 27221 | 27282 | 8.3 | 27248 | 61 | + | NSP8 |
| 31 | MN908947.3 | 27400 | 27430 | 11.3 | 27406 | 30 | + | NSP8 |
| 32 | MN908947.3 | 27674 | 27719 | 8.1 | 27691 | 45 | + | NSP8 |
| 33 | MN908947.3 | 28922 | 28932 | 5.3 | 28929 | 10 | + | NSP8 |
| 34 | MN908947.3 | 29025 | 29052 | 7.6 | 29035 | 27 | + | NSP8 |
| 35 | MN908947.3 | 29562 | 29614 | 7.6 | 29582 | 52 | + | NSP8 |
| 36 | MN908947.3 | 29626 | 29693 | 8.5 | 29656 | 67 | + | NSP8 |
| 37 | MN908947.3 | 29818 | 29868 | 6.9 | 29831 | 50 | + | NSP8 |

It was observed that only a small fraction of reads mapped to the negative sense strand in the input samples (0.00075 for N, 0.00076 for NSP 12, 0.0043 for NSP8) and the IP samples for N (0.00046). In contrast, NSP12 and NSP8 IP samples enriched the fraction of negative sense reads to 0.067 and 0.039, about 100- and 10-fold from IN samples, respectively. The negative sense strand coverage in IP samples was also lower in N (33%) than in NSP12 (80%/) or NSP8 (58%). NSP12 and NSP8 IP reads are piled up in the 5′ and 3′ regions on the negative sense strand, which is similar to the positive sense strand. The low fraction of reads in the input samples prevents further quantitative assessments. Nevertheless, the findings confirmed the roles for NSP12 and NSP8, but not N, in transcribing negative sense RNA templates to generate mRNAs for translation, and the ability for N to selectively associate with positive sense genomic RNA over negative sense RNAs.

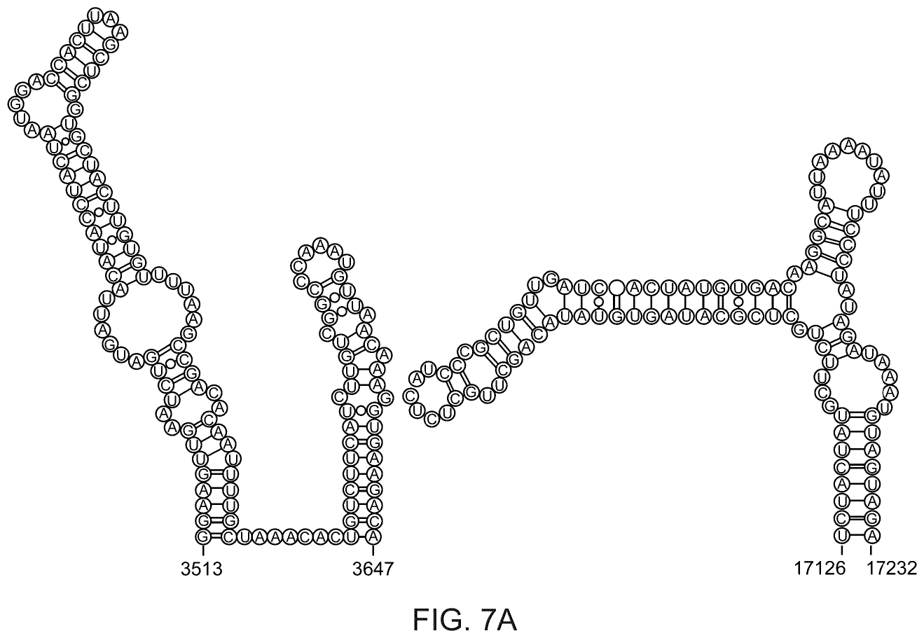

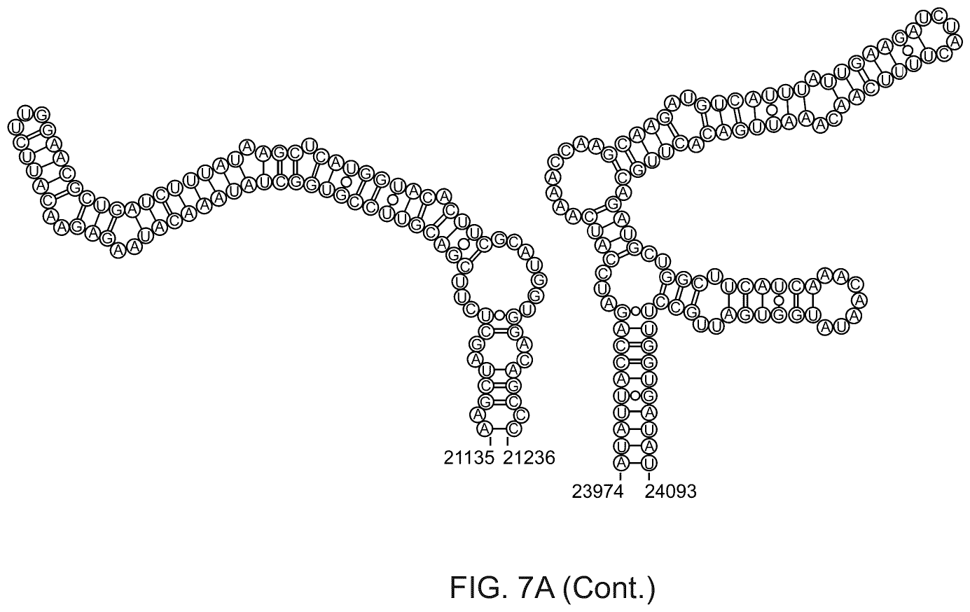

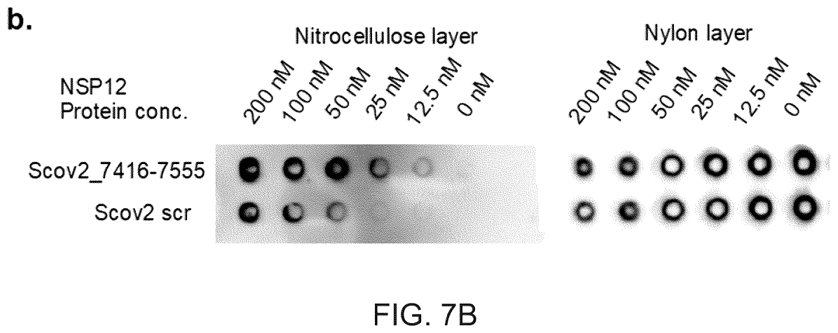

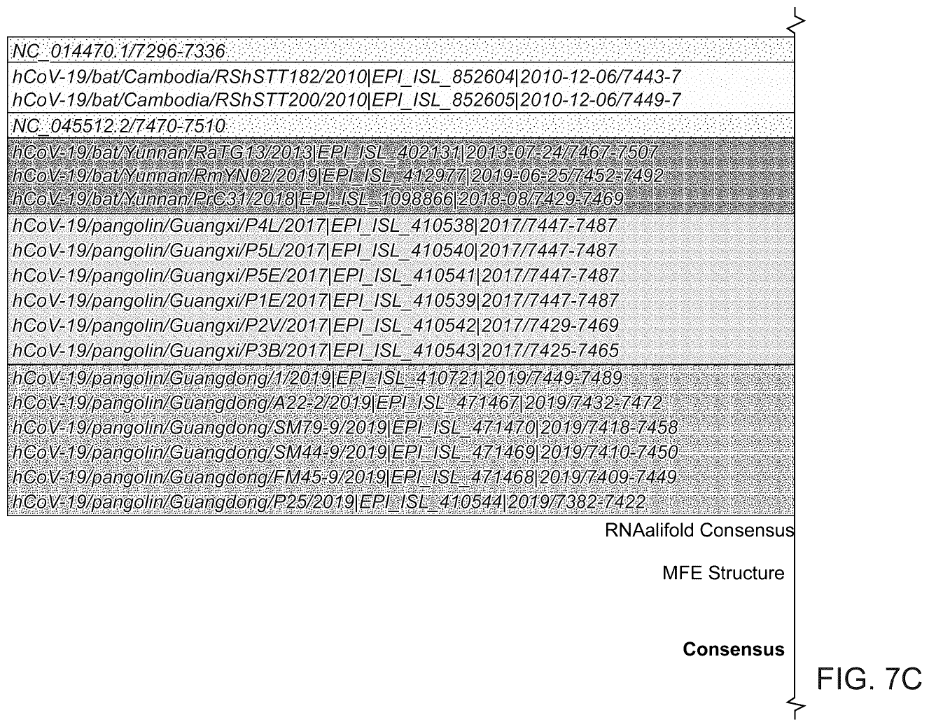

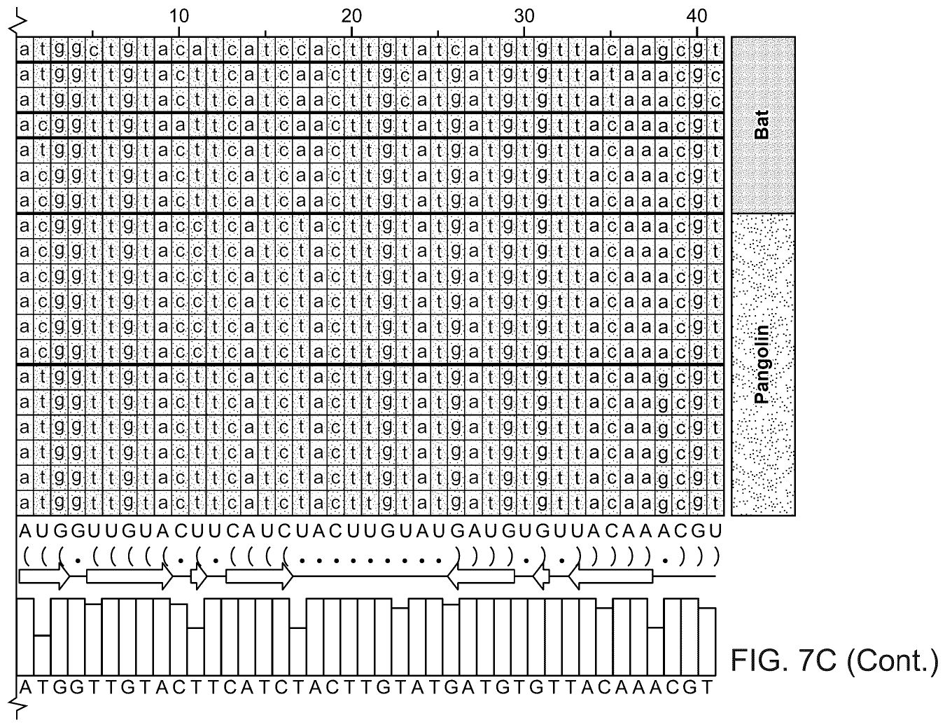

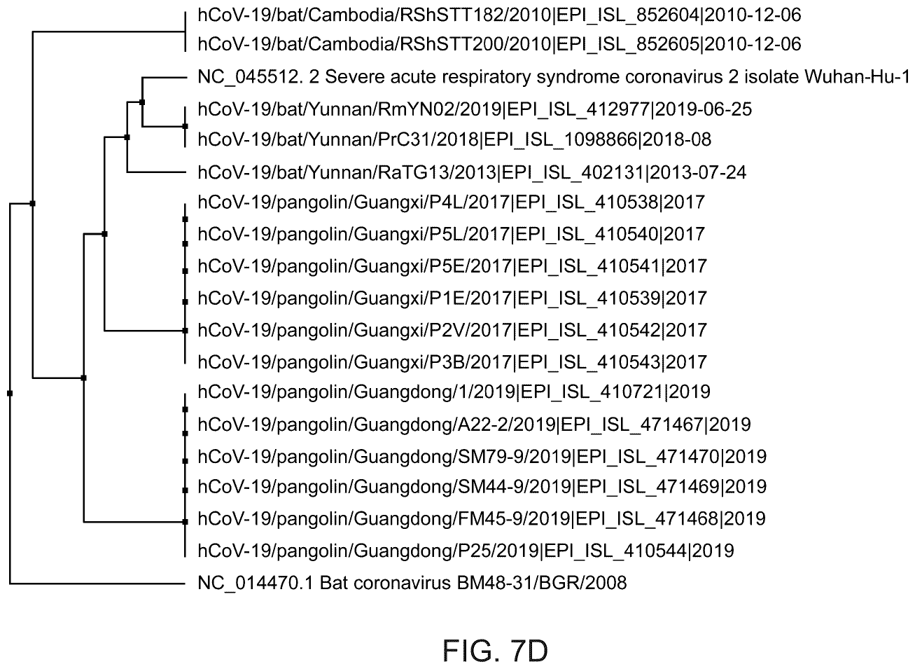

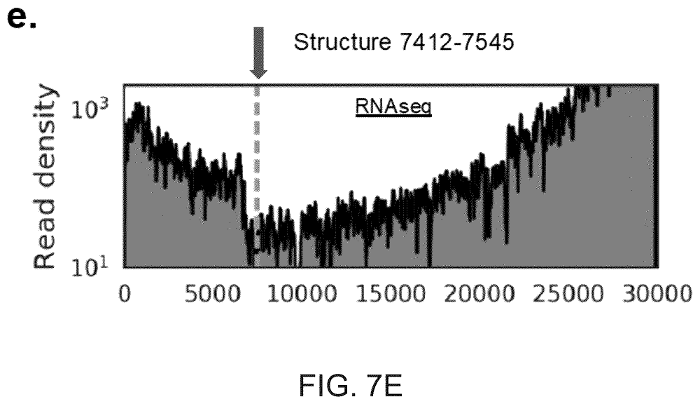

Besides regions around the 5′ and 3′ end, several sharp peaks with high relative enrichment in read density were observed (>5-fold, for a contiguous region of 10 nt or more) i.e. 22 peaks for NSP12, 37 peaks for NSP8, and 7 peaks for N (Table 2). NSP12 eCLIP had especially strong peaks; five example major peaks are highlighted (FIG. 1B-1Q) at regions 3533-3635, 7436-7526, 17202-17222, 21177-21206, and 24018-24079 (FIG. 1B), which have maximum positional fold changes of 19, 14, 9.2, 11 and 13-fold (Table 2), respectively. Intriguingly, when these regions were lined up with SHAPE reactivity data, the peaks corresponded to regions with low SHAPE Shannon entropy values, which represent regions that are rigid or structured (FIG. 1C). A closer inspection showed these stem-loop structures to have long, stable stems (FIG. 7A). As no strong sequence motifs were observed, it was hypothesized that structural elements in the SARS-CoV-2 genome likely facilitate protein-RNA interactions with NSP12. Recently, it was shown via RNA footprinting with SHAPE structure probing experiments that in addition to RNA-RNA base pairing, some nucleotides with low SHAPE reactivity may be due to direct hydrogen bond interactions with RNA binding proteins. For NSP12 and NSP8, a slight negative correlation between log 2 fold change in eCLIP read density and SHAPE entropy was further observed (FIG. 1D), where the latter correlates inversely with structuredness. The eCLIP findings can thus add a layer of functional information—interaction with NSP12—to structural elements in SARS-CoV-2.

It was noticed that the peak at position 7436-7526 is uniquely enriched in NSP12 eCLIP only. Located near the 3′ end of the gene encoding for NSP3, this peak overlapped with a structured region of 3 consecutive stem loops at region 7412-7545 (FIG. 1E). To validate the specific protein-RNA interaction, a filter binding assay of NSP12 was performed with in vitro transcribed RNA bearing the sequence in this region (FIG. 1F). A scrambled control was included with the same sequence composition but shuffled such that the structure is no longer preserved (Table 3). The peak RNA showed a binding dissociation constant K_{D} of 17 nM, compared to a K_{D} of 79 nM by the scrambled control (FIG. 1G). The scrambled control likely represents the non-specific affinity of NSP12 for RNA (FIG. 7B). The sequence and structure of the central hairpin, where the highest point of the peak fold change is located, were also highly conserved among related betacoronaviruses (FIGS. 7C-7D). It also appeared to be located ˜500 nt downstream of the steplike reduction in RNA reads extending from the 5′ side of the genome (FIG. 7E), though the functional linkage between the two features is unknown.

TABLE 3
Nucleic acid sequences used in filter binding assay

| Name | Type | Sequence 5′→3′ | SEQ ID NO |
|---|---|---|---|
| T7_fwd | Single stranded DNA | TAATACGACTCACTATAGGGCGAAAACGCCC | 41 |
| | oligonucleotide | | |
| Scov2_7431_7555_ | Single stranded DNA | ACTATAGGGCGAAAACGCCC | 42 |
| left | oligonucleotide | CATTTTATTATGTATGGAAAAGTTATGTGCATGTTGTAGACGGTTGTAATTCATCAACTTGTATGATG | |
| Scov2_7431_7555_ | Single stranded DNA | AACACCATTAACAATAGTTGTACATTCGACTCTTGTTGCTCTATTACGTTTGTAACACATCATACAAGTT | 43 |
| right_revcomp | oligonucleotide | GATGAATTACAACCG | |
| Scov2_7431_7555_ | Single stranded DNA | AACACCATTAACAATAGTTGTACATTCG | 44 |
| rev | oligonucleotide | | |
| Scov2_7431_7555_ | Single stranded DNA | ACTATAGGGCGAAAACGCCC | 45 |
| Scrambled_left | oligonucleotide | GAATTTTATCATGACTGAACTGTTTTTGTGTCCCAAAAATAGTGCTACATTAGGAAAATAATTAAGATTA | |
| Scov2_7431_7555_ | Single stranded DNA | CCCTCCTGATAAGTAAACCCTAGTACTAAATAACATATTAACTCCAATTCAGCCGTAATCTTAATTATTT | 46 |
| Scrambled_right_ | oligonucleotide | TCCTAATGTAGCACTATTTT | |
| revcomp | | | |
| Scov2_7431_7555_ | Single stranded DNA | CCCTCCTGATAAGTAAACCCTAG | 47 |
| Scrambled_reV | oligonucleotide | | |
| Scov2_7431_7555_ | Double stranded DNA, PCR | TAATACGACTCACTATAGGGCGAAAACGCCCCATTTTATTATGTATGGAAAAGTTATGTGCATGTTGTAG | 48 |
| PCR | product | ACGGTTGTAATTCATCAACTTGTATGATGTGTTACAAACGTAATAGAGCAACAAGAGTCGAATGTACAAC | |
| | | TATTGTTAATGGTGTT | |
| Scov2_7431_7555_ | Double stranded DNA, PCR | TAATACGACTCACTATAGGGCGAAAACGCCCACTATAGGGCGAAAACGCCC | 49 |
| Scrambled_PCR | product | GAATTTTATCATGACTGAACTGTTTTTGTGTCCCAAAAATAGTGCTACATTAGGAAAATAATTAAGATTA | |
| | | CGGCTGAATTGGAGTTAATATGTTATTTAGTACTAGGGTTTACTTATCAGGAGGG | |
| Scov2_7431_7555_ | In vitro transcribed RNA | GGGCGAAAACGCCCCAUUUUAUUAUGUAUGGAAAAGUUAUGUGCAUGUUGUAGACGGUUGUAAUUCAUCA | 50 |
| RNA | | ACUUGUAUGAUGUGUUACAAACGUAAUAGAGCAACAAGAGUCGAAUGUACAACUAUUGUUAAUGGUGUU | |
| Scov2_7431_7555_ | In vitro transcribed RNA | GGGCGAAAACGCCCGAAUUUUAUCAUGACUGAACUGUUUUUGUGUCCCAAAAAUAGUGCUACAUUAGGAA | 51 |
| Scrambled_RNA | | AAUAAUUAAGAUUACGGCUGAAUUGGAGUUAAUAUGUUAUUUAGUACUAGGGUUUACUUAUCAGGAGGG | |

Finally, the relative eCLIP enrichment between NSP8, NSP12 and N was compared to investigate any similarities in the positive sense RNA interaction between the three proteins. When comparing the relative positional enrichment, N and NSP12 show no correlation (R² of 0.01), whereas N and NSP8 are slightly correlated (R2 of 0.27; FIG. 11H). As expected, NSP8 and NSP12 were the most highly correlated (R2 of 0.57), though there were still substantial differences, as 43% of the variation was unaccounted for by the other protein (FIG. 11H). However, when the data was transformed logarithmically, greater correlations among all three proteins were observed. As logarithmic transformations shed light on signals at lower values, the greater correlations among the log-transformed data of different proteins may imply a greater similarity of more transient protein-RNA interactions. This invites future inquiry about the importance of transient protein-RNA interactions in the life cycle of SARS-CoV-2.

To evaluate the blocking of the NSP12 binding to SARS-CoV-2 RNA from position 7414-7555, or disruption of the structure, a locked nucleic acid (LNA) was designed to be complementary to the middle hairpin, specifically position 7472-7493 (FIG. 1I). LNA7472-7493 (SEQ ID NO: 52—+T+A+C+A+A+GT+T+G++AT+G3A+A+T+TA+C+A+A+C+C) and scrambled LNA (SEQ ID NO: 53—+G+C+GGC+ACG+TTG+CG+AGT+A+C+T) were synthesized by Integrated DNA Technologies. The LNA was predicted to hybridize with SARS-CoV-2 RNA from position 7414-7555 using the NUPACK software1 (FIG. UJ). Vero E6 cells transfected with LNA7472-7493 and a scrambled LNA were infected with SARS-CoV-2 (WA1 isolate) and the infection rate was assessed by immunofluorescence 24 hours post infection. It was found that treating cells with LNA7472-7493 reduced the infection rate (as given by integrated intensity) by 64% (p<0.01) (FIGS. 1K-1L). This shows that targeting structured genomic RNA interacting with SARS-CoV-2 protein NSP12 may be a potential antiviral treatment strategy. For therapeutic development, modalities may not be limited to LNAs, but also to include siRNAs and small molecules.

### Example 2—SARS-CoV-2 Proteins Interact with Host RNAs in Virus-Infected Cells

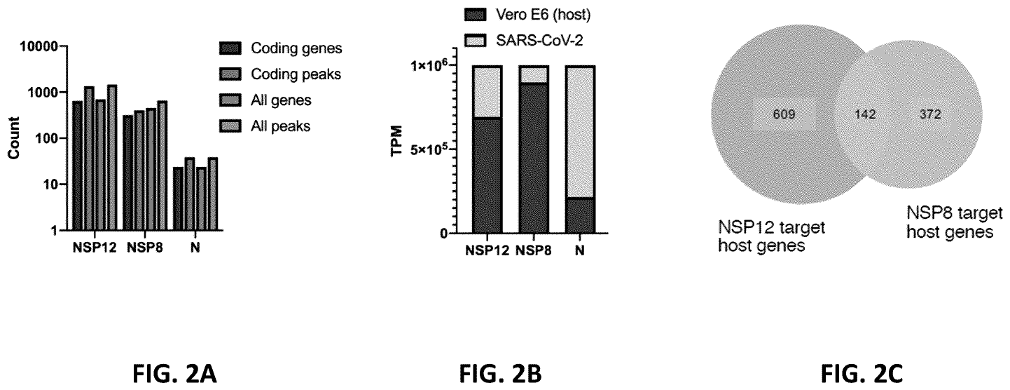

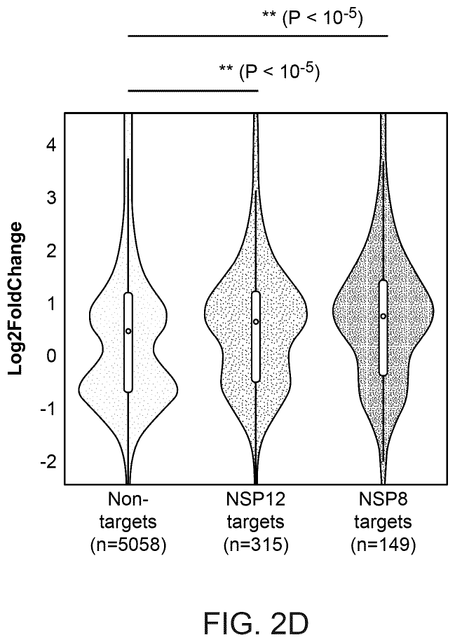

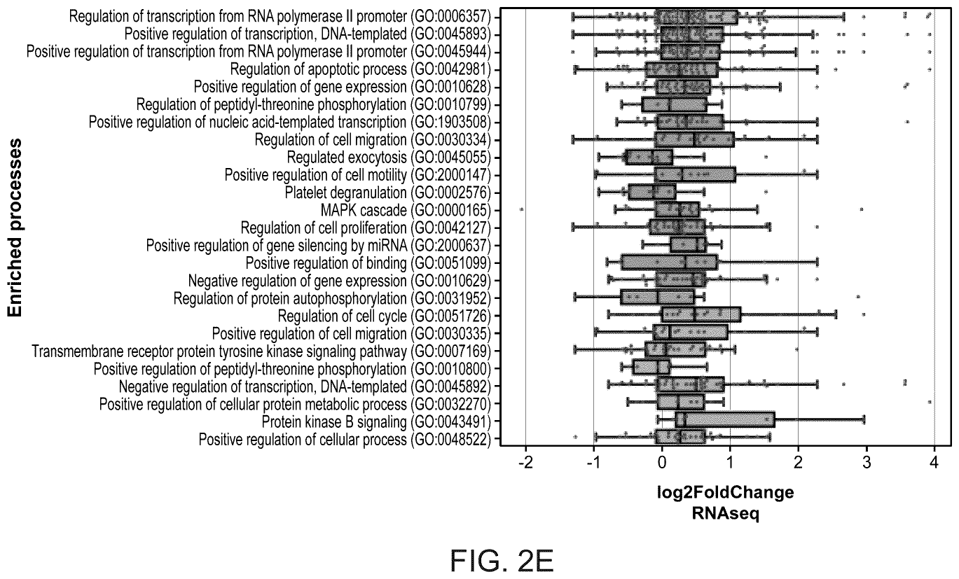

As RNA virus infections have been shown to perturb host transcriptomes, such as via mRNA degradation, mRNA export inhibition, splicing interference, 5′ cap stealing, and other ways of host translation inhibition, evidence for direct interaction between viral proteins host cell RNAs can shed light on the mechanism and function as a result of these interactions. Therefore, the extent to which NSP8, NSP12 and N interacted with host RNAs was investigated. Targeted transcripts were determined by having one or more peaks that meet the stringent IDR (irreproducible discovery rate) threshold of overlapping peaks between two replicates for every protein, and satisfy statistical cutoffs of p<0.001, and more than 8-fold enrichment in the immunoprecipitated sample (IP) over the size-matched input sample. It was found that NSP8, NSP12 and N interact with 457, 703 and 24 genes with 658, 1457 and 39 significant peaks, respectively (FIG. 2A). Interestingly, the number of RNA reads in Transcripts Per Kilobase Million (TPM) from both NSP8 and NSP12 immunoprecipitation (IP) samples were mapped more frequently to host transcripts than viral RNA (FIG. 2B). Among the target genes, NSP12 and NSP8 shared 128 genes in common (18% of NSP12 targets, 26% of NSP8 targets), implying that NSP12 and NSP8 may interact with different host genes in both their individual and complexed states (FIG. 2C). In contrast, a majority of N immunoprecipitated RNA reads were mapped to viral RNA, consistent with its role in enclosing the viral genome during virion assembly. The large number of peaks (2137 total) that map to the 1058 host genes further suggests a potential in perturbing host gene expression that may be required for viral replication.

To determine if there are differences in expression levels of the host target genes whose mRNAs are enriched in NSP12 and NSP8 eCLIP, transcriptome-wide mRNA sequencing of SARS-CoV-2 infected Vero E6 cells was performed and the eCLIP target genes were mapped to differential expression levels. It was found that NSP12 and NSP8 target mRNA levels are significantly increased than non-target genes (p<10⁻⁵, KS test; FIG. 2D). To understand the processes enriched by the target genes, a Gene Ontology analysis was performed. 54 GO processes that are significantly enriched by NSP12 target genes were found (P_{adjusted}<0.01) (Table 4), whereas no significant GO processes are found in NSP8 target genes. Many of the GO processes fall into three broad categories related to regulating transcription and gene expression, cell cycle and apoptosis, and phosphorylation and signaling processes (FIG. 2E). Of the transcription regulation genes, many have antiviral response properties (e.g. NF-κB, BATF, NR4A1, BMP2, SQSTM1, MAFF, MDM2), while others have demonstrated proviral activities, such as DDX5, SFPQ, FBXW11 and ATF-3. Of the genes regulating cell proliferation, cell cycle and apoptosis, PAK2 has been associated with anti-apoptotic signaling and promoting HIV survival, whereas many other genes have overlapping annotation as the transcription regulation genes. A recent study elucidated global phosphorylation changes in cellular proteins upon SARS-CoV-2 infection. Specifically, the p38/MAPK cascade activity is induced by viral infection, and treatment with p38 inhibitors has restrictive effects on viral proliferation. In the NSP12 eCLIP data, enrichment of the MAPK cascade and other signaling pathway genes was also shown (e.g. MAPK1, MAP2K1/3, MAP4K4/5, PIM3, PAK2, EPHA2). In the context of the whole virus infection where a multitude of viral proteins and host defense responses are at play, it cannot be definitively concluded that the interactions between NSP12 and these mRNAs have a causative or inhibitive relationship. Nevertheless, the correlation of NSP12 protein-RNA interactions with these pathway genes, which are relevant to viral infection and host response, leads to hypothesize a potential, albeit unknown, role, and the data represents a rich resource for subsequent mechanism studies. To understand the individual contribution of viral protein-host RNA interactions, the study proceeded to profile the protein-RNA interactions of each SARS-CoV-2 protein.

TABLE 4
Enriched Gene Ontology processes in NSP12 eCLIP in SARS-CoV-2 infected cells

| | | Adjusted |
|---|---|---|
| | Gene Ontology Term | P-value |
| 1 | regulation of transcription from RNA polymerase II promoter (GO:0006357) | 1.3E−08 |
| 2 | positive regulation of transcription, DNA-templated (GO:0045893) | 6.9E−08 |
| 3 | positive regulation of transcription from RNA polymerase II promoter (GO:0045944) | 2.8E−06 |
| 4 | regulation of apoptotic process (GO:0042981) | 2.8E−06 |
| 5 | positive regulation of gene expression (GO:0010628) | 5.7E−05 |
| 6 | regulation of peptidyl-threonine phosphory lation (GO:0010799) | 2.6E−04 |
| 7 | positive regulation of nucleic acid-templated transcription (GO:1903508) | 4.2E−04 |
| 8 | regulation of cell migration (GO:0030334) | 5.4E−04 |
| 9 | regulated exocytosis (GO:0045055) | 5.4E−04 |
| 10 | positive regulation of cell motility (GO:2000147) | 6.1E−04 |
| 11 | platelet degranulation (GO:0002576) | 6.2E−04 |
| 12 | MAPK cascade (GO:0000165) | 7.8E−04 |
| 13 | regulation of cell proliferation (GO:0042127) | 7.8E−04 |
| 14 | positive regulation of gene silencing by miRNA (GO:2000637) | 8.7E−04 |
| 15 | positive regulation of binding (GO:0051099) | 1.2E−03 |
| 16 | negative regulation of gene expression (GO:0010629) | 1.2E−03 |
| 17 | regulation of protein autophosphorylation (GO:0031952) | 1.9E−03 |
| 18 | regulation of cell cycle (GO:0051726) | 1.9E−03 |
| 19 | positive regulation of cell migration (GO:0030335) | 2.7E−03 |
| 20 | transmembrane receptor protein tyrosine kinase signaling pathway (GO:0007169) | 2.8E−03 |
| 21 | positive regulation of peptidy1-threonine phosphorylation (GO:0010800) | 3.0E−03 |
| 22 | negative regulation of transcription, DNA-templated (GO:0045892) | 3.0E−03 |
| 23 | positive regulation of cellular protein metabolic process (GO:0032270) | 3.0E−03 |
| 24 | protein kinase B signaling (GO:0043491) | 3.5E−03 |
| 25 | positive regulation of cellular process (GO:0048522) | 3.5E−03 |
| 26 | positive regulation of cell proliferation (GO:0008284) | 3.5E−03 |
| 27 | regulation of fibroblast proliferation (GO:0048145) | 3.7E−03 |
| 28 | negative regulation of programmed cell death (GO:0043069) | 3.7E−03 |
| 29 | negative regulation of apoptotic process (GO:0043066) | 3.7E−03 |
| 30 | regulation of transcription, DNA-templated (GO:0006355) | 3.7E−03 |
| 31 | stress-activated protein kinase signaling cascade (GO:0031098) | 3.7E−03 |
| 32 | negative regulation of transcription from RNA polymerase II promoter (GO:0000122) | 3.7E−03 |
| 33 | positive regulation of protein phosphorylation (GO:0001934) | 3.8E−03 |
| 34 | cell-cell junction organization (GO:0045216) | 4.4E−03 |
| 35 | positive regulation of protein kinase activity (GO:0045860) | 4.4E−03 |
| 36 | cellular response to transforming growth factor beta stimulus (GO:0071560) | 5.2E−03 |
| 37 | transmembrane receptor protein serine/threonine kinase signaling pathway (GO:0007178) | 5.3E−03 |
| 38 | mRNA 3′-end processing (GO:0031124) | 5.3E−03 |
| 39 | ERK1 and ERK2 cascade (GO:0070371) | 5.8E−03 |
| 40 | regulation of cell-matrix adhesion (GO:0001952) | 6.9E−03 |
| 41 | regulation of stress-activated MAPK cascade (GO:0032872) | 6.9E−03 |
| 42 | transcription, DNA-templated (GO:0006351) | 6.9E−03 |
| 43 | regulation of mitotic cell cycle (GO:0007346) | 6.9E−03 |
| 44 | positive regulation of protein autophosphorylation (GO:0031954) | 6.9E−03 |
| 45 | regulation of programmed cell death (GO:0043067) | 7.6E−03 |
| 46 | branching morphogenesis of an epithelial tube (GO:0048754) | 8.3E−03 |
| 47 | regulation of cell-substrate junction assembly (GO:0090109) | 8.3E−03 |
| 48 | negative regulation of RNA metabolic process (GO:0051253) | 8.3E−03 |
| 49 | cellular response to corticosteroid stimulus (GO:0071384) | 8.3E−03 |
| 50 | negative regulation of peptidyl-threonine phosphorylation (GO:0010801) | 8.3E−03 |
| 51 | positive regulation of intracellular signal transduction (GO:1902533) | 8.3E−03 |
| 52 | regulation of cell motility (GO:2000145) | 8.8E−03 |
| 53 | regulation of focal adhesion assembly (GO:0051893) | 8.9E−03 |
| 54 | transcription from RNA polymerase II promoter (GO:0006366) | 9.9E−03 |

### Example 3—Exogenously Expressed SARS-CoV-2 Proteins Interact with One Third of the Transcriptome in Lung Epithelial Cells

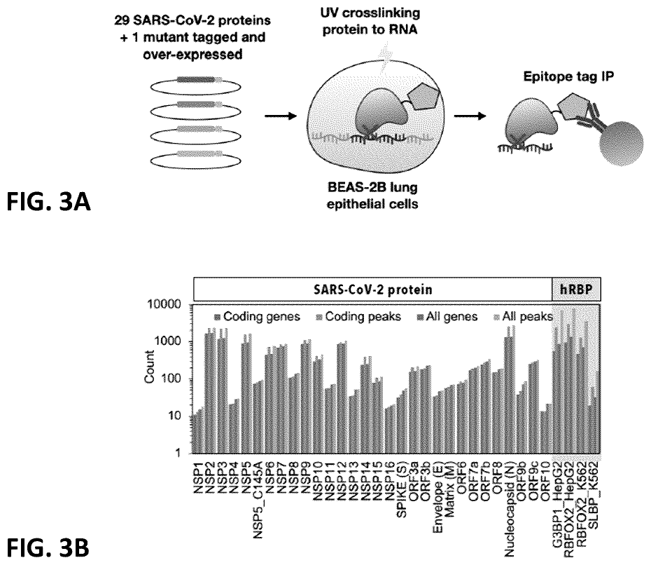

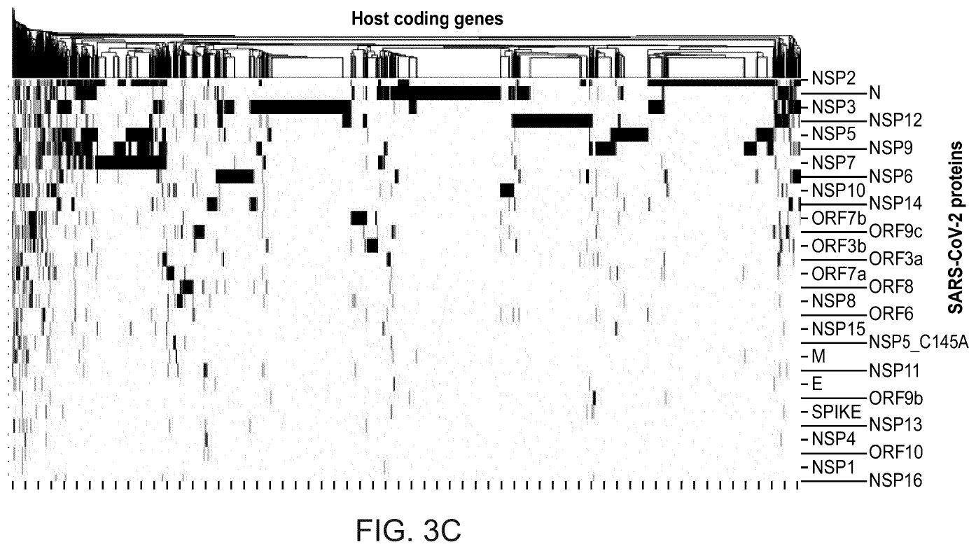

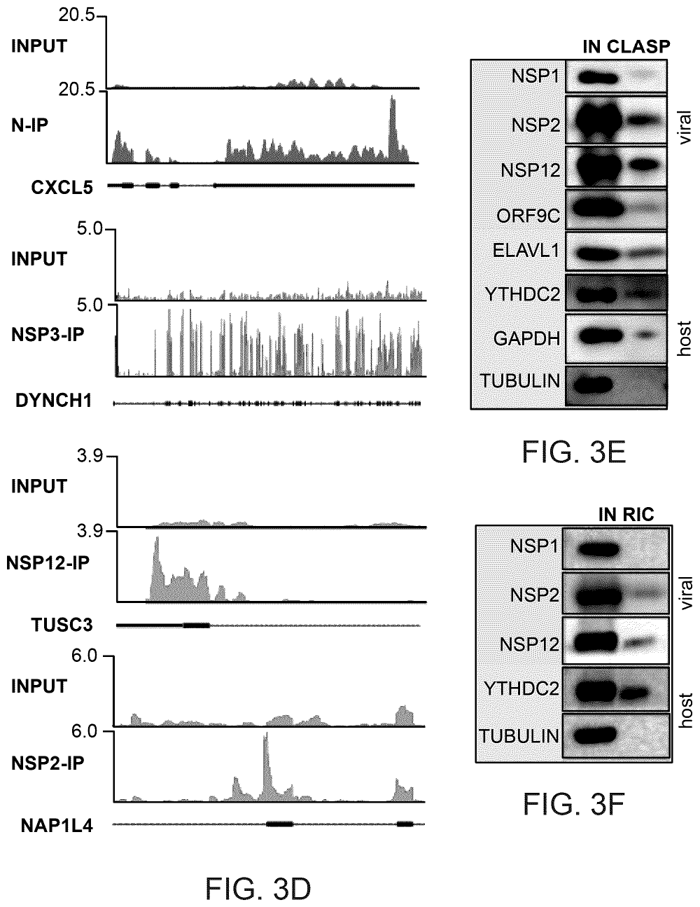

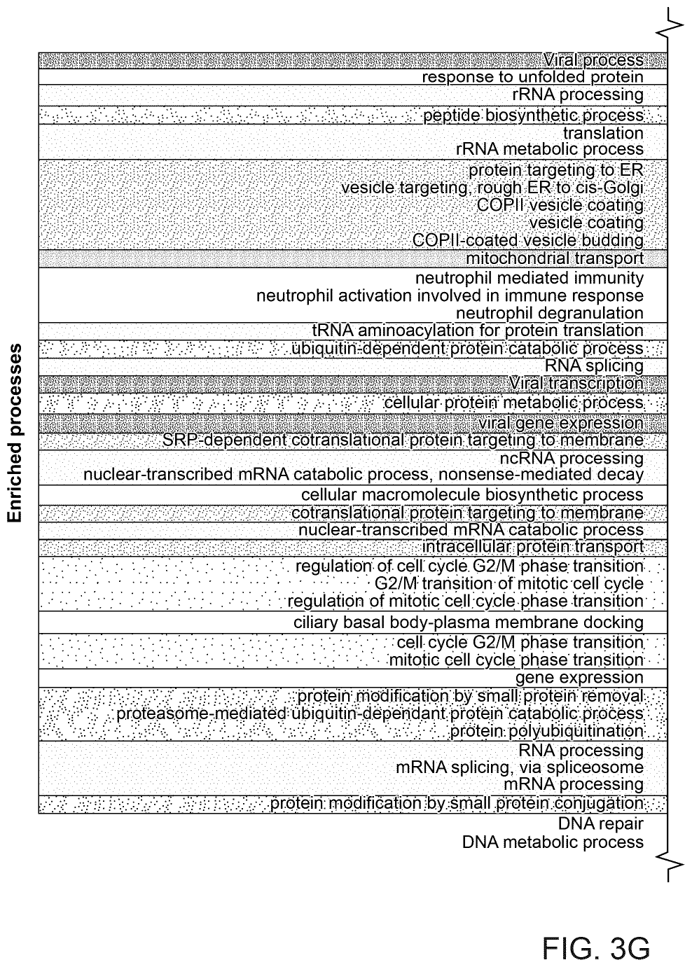

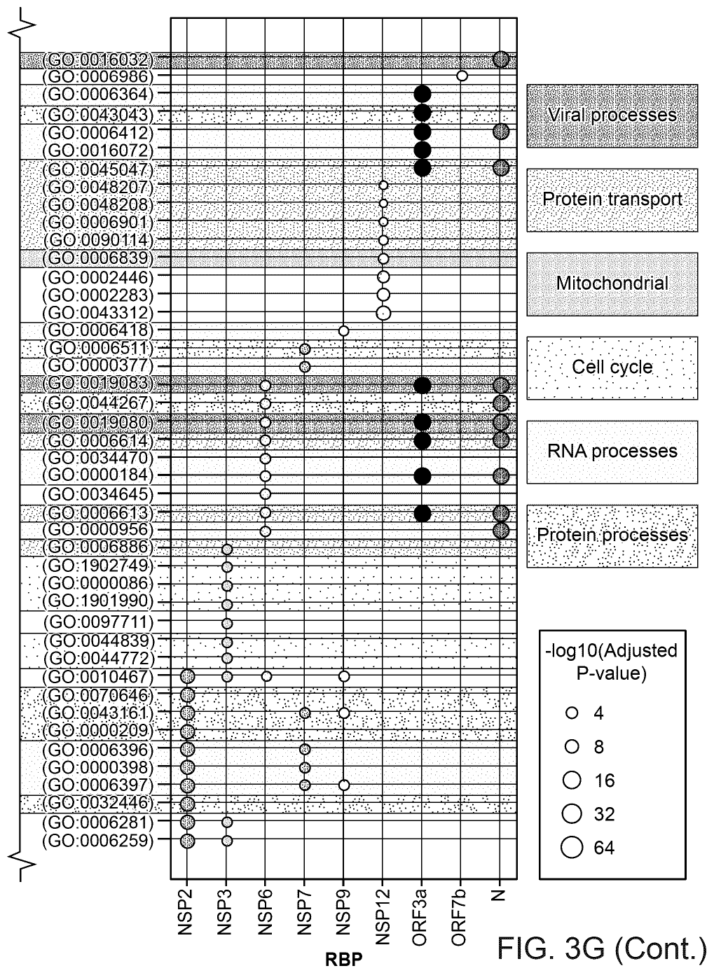

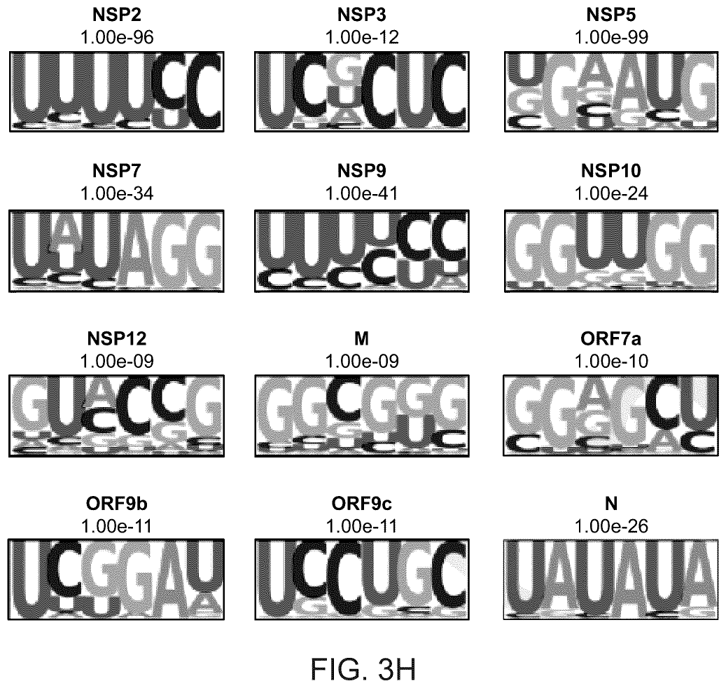

Even though NSP12, NSP8, and N eCLIPs were performed in virus infected vero cells, in order to further investigate whether SARS-COV-2 proteins directly interact with the human host transcriptome, eCLIP was performed on the 29 proteins encoded in the SARS-CoV-2 genome and one mutant (FIG. 3A). Due to the lack of antibodies specific for most of the viral proteins, the individual proteins were exogenously expressed in a lung epithelial cell line BEAS-2B, which is an immortalized primary bronchial cell line representative of normal lung physiology. Each protein was either fused with a 2×Strep tag and expressed stably via lentiviral transduction or fused with a 3×FLAG tag and expressed transiently via transfection. Following UV crosslinking, the tagged proteins were immunoprecipitated using anti-FLAG or anti-Strep antibodies. Subsequent RNA purification and library purification steps were performed as in the viral eCLIP experiments. Cells expressing only the 3×FLAG or 2×Strep tags and wildtype cells were used as controls to remove background peaks in subsequent analysis steps.

From the SARS-CoV-2 proteome-wide eCLIP results, SARS-CoV-2 proteins interacted with RNA represented by 4773 coding genes, which is about a third of the transcriptome of BEAS-2B cells. Nucleocapsid and non-structural proteins NSP2, NSP3, NSP5, NSP9 and NSP12 were found to target the greatest number of unique genes at 1339, 1647, 1199, 902, 863, and 865, respectively (FIG. 3B). The large number of genes targeted by the viral proteins is consistent with the non-structural proteins from the replicase (ORF1ab) having a high affinity for its own RNA, though their potential for widespread interaction with host RNA has not been shown previously.

The widespread interaction of Nucleocapsid with host RNAs when expressed in isolation is consistent with its capacity for nonspecific RNA binding, whereas its targeting the virus genome during RNA assembly occurs via interaction with the M protein. For comparison, the extensively studied splicing factor RBFOX2 binds to 958 genes in HepG2 cells and 471 genes in K562 cells, the stress granule assembly factor G3BP1 binds to 561 genes in HepG2 cells, and the histone RNA hairpin-binding protein SLBP binds to 19 genes in K562 (FIG. 3B). This suggests that viral proteins have similar capacities for interacting with RNA as endogenous human RBPs.

Target genes with at least one significant eCLIP peak also appear highly distinct across the different SARS-CoV-2 proteins (FIG. 3C). Within individual targets, eCLIP reads also display different profiles, example include N eCLIP peak in 3′ UTR of CXCL1, NSP3 peaks found across all exons in DYNCH1, a NSP12 peak in 5′ UTR of TUSC3 and a NSP2 peak in the intronic region upstream of 3′ splice site of NAP1L4 (FIG. 3D). To cross validate the eCLIP findings that SARS-CoV-2 proteins interact with host cell RNAs, a subset of these proteins were validated as RBPs using crosslinking and solid phase purification (CLASP26), which stringently captures crosslinked protein-RNA interactions due to denaturing wash conditions. HEK293T cells transiently expressing NSP1, NSP2, NSP12, and ORF9c followed by pulldown of total RNA showed enrichment of these proteins (FIG. 3E). For comparison, host RNA binding proteins ELAVL1, YTHDC1 and GAPDH were also included as positive controls, and tubulin as negative controls. Furthermore, RNA interactome capture (RIC27) of poly-A RNA (mRNA, lincRNA, and other POLII transcripts) pulldown was performed using an oligo (dT) primer and found that NSP2 and NSP12 were enriched, but not NSP1 (FIG. 3F), which mostly enriched ribosomal RNAs in eCLIP.

Distinct processes related to viral replication and host response are targeted by the viral proteins as shown by gene ontology (GO) analysis (FIG. 3G). Many of the enriched GO terms are related to nucleic acid and protein synthesis, modification and transport, which is consistent with the primary objective of the virus hijacking host resources for its own biosynthesis and replication. Notably, several protein transport processes are enriched, namely SRP-dependent protein targeting to membrane as enriched by NSP6, ORF3a and N, and COPII vesicle budding and targeting from rough ER to Golgi as enriched by NSP12. These may be involved in viral vesicle formation to serve as replication organelles, as found in a number of positive sense RNA viruses. Immune response processes are also enriched, including neutrophil mediated immunity targeted by NSP12 and platelet degranulation targeted by ORF9c. This supports the choice of lung epithelial cells as a model system that express the relevant cytokines for recruiting immune cells. While the enriched GO terms are highly relevant to viral and host response processes, further analysis of binding patterns is needed to determine if there are any functional implications of viral proteins interacting with these genes.

To determine if there are sequence features that the viral proteins recognize, sequence logos from 6-mers of eCLIP peaks were generated. While some of the proteins display strong sequence preferences, most proteins appear to interact more non-specifically. Some motifs resemble enrichments observed for human RBPs, where M, ORF7a and NSP10 appear to favor G-rich or GU rich motifs, and NSP5 has a motif (GNAUG). Other motifs may result from regional binding preferences (FIG. 3H), as NSP2 and NSP9 have a strong preference for UC-rich polypyrimidine motifs (p values of 10⁻⁹⁶ and 10⁻⁴¹ respectively), which may be a result of their binding to polypyrimidine tracts in intronic regions, whereas N has an AU-rich motif likely because it preferentially binds to 3′ UTR which contain AU-rich elements. NSP3, a large multifunctional protein, appears to coat entire exons and may not have a meaningful sequence motif. NSP12 primarily binds in the 5′ UTR, and a weakly enriched GUCCCG motif that resembles terminal oligopyrimidine (TOP) motifs hints at a possible role in translation perturbation.

The systematic interrogation of SARS-CoV-2 protein-host RNA interactions demonstrates that a majority of SARS-CoV-2 viral proteins are RNA binding proteins that target roughly a third of the human transcriptome. This analysis implies that these viral proteins may be involved in perturbing many essential cellular processes of the host. As eCLIP in virus infected cells are limited by IP-grade antibodies, the data obtained from the exogenous expression of individual proteins in BEAS-2B cells was used for systematic analysis of potential functional implications.

### Example 4—SARS-CoV-2 Proteins Upregulate Protein Expression of Target Transcripts

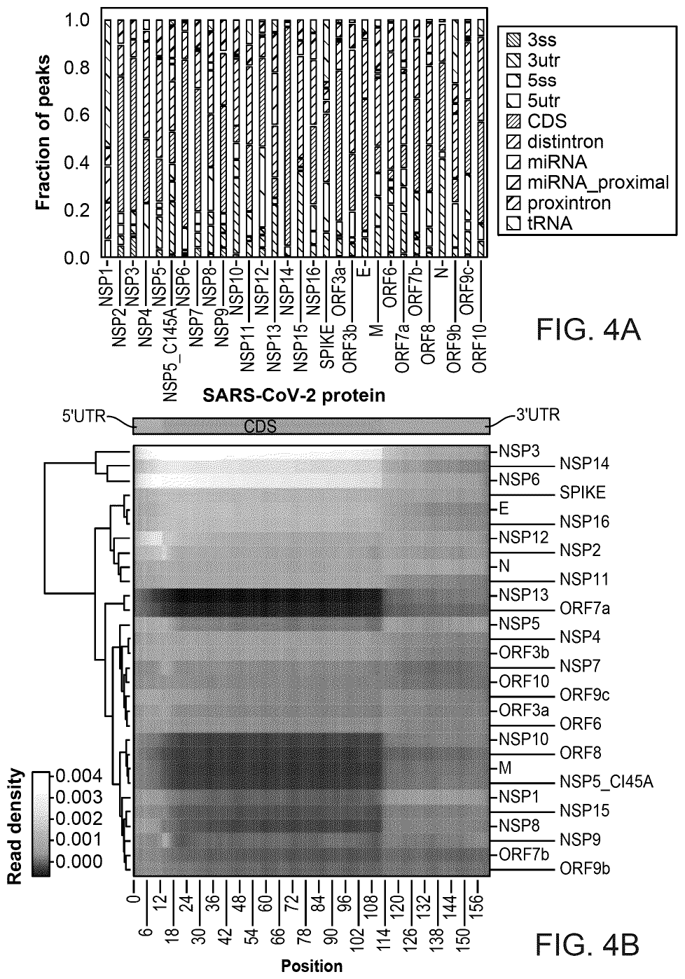

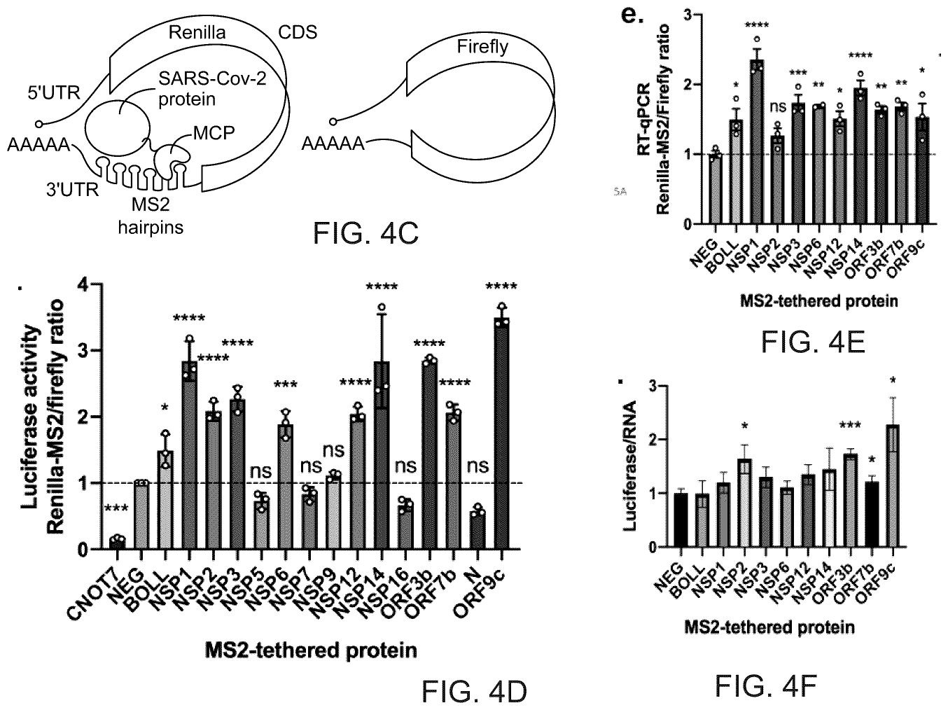

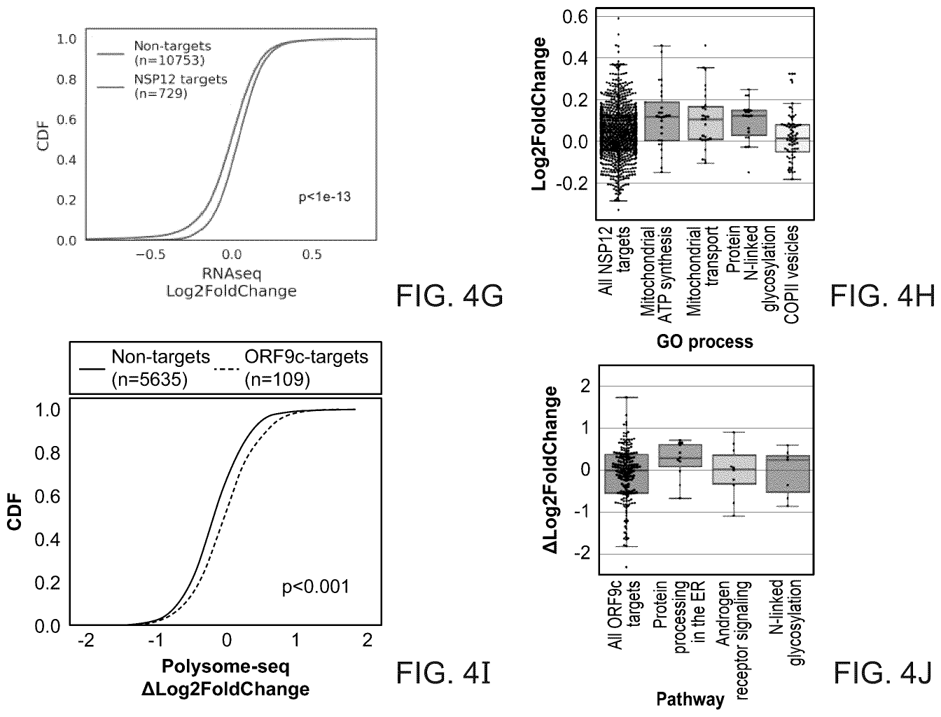

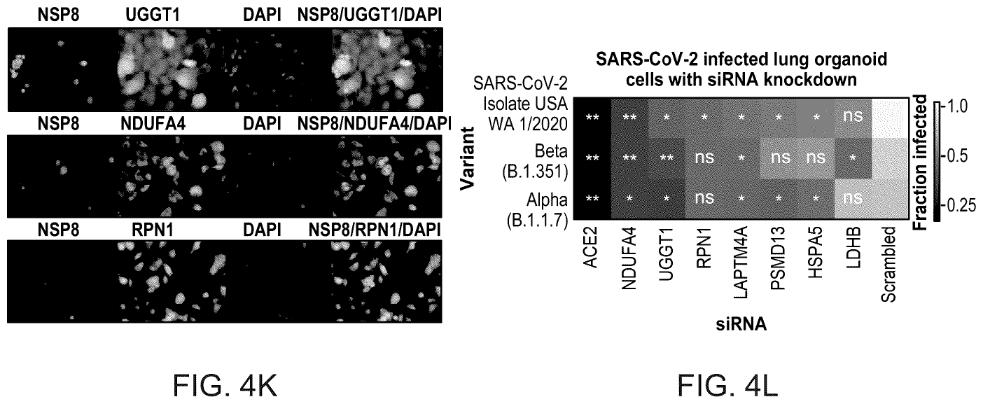

By examining the regional binding preferences of each SARS-CoV-2 protein, it was found that SARS-CoV-2 proteins are enriched at distinct regions of target mRNAs, which imply different regulatory functions because of the protein-RNA interaction. Aggregating the analysis of all targeted peaks for each SARS-CoV-2 protein identifies RNA regions that are preferentially bound (FIG. 4A). Of note, NSP12, ORF3b, ORF7b and ORF9c show the highest proportion of peaks in the 5′ UTR; NSP2, NSP3, NSP6 and NSP14 show the highest proportion of peaks in the coding region (CDS), NSP5, NSP7 and NSP9 display a high proportion of peaks in intronic regions, and N and NSP15 show the largest proportion of peaks in the 3′ UTR. Also, a metagene analysis of read density was performed across all target mRNA transcripts, where each of the 5′ UTR, CDS and 3′ UTR regions in an mRNA are scaled to standardized lengths (FIG. 4B). It was found that even though NSP2 has a similar number and proportion of peaks in the CDS as NSP3, it mainly targets the region spanning the 5′ UTR and coding start. In contrast, NSP3 reads, along with that of NSP6 and NSP14, coat the entire CDS, with a slight bias towards the start of the coding sequence.

Since 8 of the SARS-CoV-2 proteins—NSP2, NSP3, NSP6, NSP12, NSP14, ORF3b, ORF7b and ORF9c—have binding preferences at the 5′ UTR and CDS, it was hypothesized that their protein-RNA interactions could affect expression of the target mRNAs at the level of RNA turnover or translation. To evaluate the functional role of the specific protein-RNA interactions of SARS-CoV-2 proteins and target transcripts, 14 of the proteins with the highest number of unique target coding genes were characterized using tethered function reporter assays (FIG. 4C). The individual proteins were fused with an MS2 phage coat protein (MCP), which localizes the tagged protein to MS2 aptamer hairpins inserted in the 3′ UTR of Renilla luciferase. A firefly luciferase without MS2 hairpins is included as a control for non-specific effects of the viral protein. Plasmids encoding the MCP-tagged proteins and reporter constructs were co-transfected into HEK293T cells. Changes in Renilla luciferase activity normalized to firefly luciferase activity measures up- or downregulation of protein expression via either translation or mRNA stability because of positioning the MCP tagged protein in the vicinity of the Renilla mRNA. The luciferase readout does not by itself distinguish between translational or mRNA stabilizing effects.

From the tethering experiments, it was found that the ratio of Renilla-MS2 to firefly luciferase for 9 of the 14 SARS-CoV-2 proteins increase 1.9 (NSP6) to 3.5-fold (ORF9c) relative to FLAG-MCP control (p-value <0.002, two tailed multiple t-test) (FIG. 4D). Interestingly, these SARS-CoV-2 proteins raise the target luciferase activity to greater extent than the tethering of BOLL (1.5-fold), which is a human RBP previously characterized to be amongst the strongest up-regulators from a screen of more than 700 human RBPs. Even though NSP1 eCLIP enriched very few host mRNAs and its peaks are not mapped to the 5′ UTR and CDS, these results for NSP1 are consistent with its ability to enhance the translation of its own mRNA via interacting with the 5′ UTR of the genomic viral mRNA. Of the remaining 5 SARS-CoV-2 proteins, only NSP5, NSP16 and N display slight (but not significant) down-regulation effects (0.73-fold to 0.58-fold) compared to the FLAG peptide control, but to a lesser extent than that of the known translation repressor CNOT7 (0.16-fold). NSP7 and NSP9 appear to have no effect on the relative luciferase activity of the target Renilla reporter. To understand if the increase in luciferase activity is occurring at the RNA or protein level, RT-qPCR was performed to measure the ratio of Renilla-MS2 to Firefly mRNAs. For all the proteins except for NSP2, the Renilla-MS2/Firefly mRNA ratio is significantly increased (p<0.05) compared to wildtype, albeit to different extents for different proteins (FIG. 4E). Of note, ORF9c shows the greatest enhancing effect (3.5-fold) in the dual luciferase assay, but its effect on the reporter RNAs is middling (1.5-fold). However, ORF9c displays the greatest fold change in luciferase activity ratio to RNA ratio (2.3-fold) (FIG. 4F), followed by NSP2 and ORF3b (1.6 and 1.7 fold respectively). The rest of the proteins range from 1.1-fold (NSP6) to 1.5-fold (NSP14), compared to 1.0-fold of BOLL, suggesting that the increase in abundance of the targeted reporter likely occurs at both the RNA and protein levels.

Based on the results of the reporter assay, it was hypothesized that SARS-CoV-2 proteins that interact with the 5′ UTR and CDS of target genes could increase their abundance. Since NSP12 demonstrated targeted increase of reporter mRNA levels, NSP12 was transiently overexpressed and mRNA sequencing was performed to determine if there are transcriptome-wide changes in gene expression. By comparing HEK293T cells transfected with NSP12 versus cells transfected with an empty plasmid vector, it was observed that the eCLIP targets of NSP12 have greater log 2 fold changes in mRNA levels than genes that are not eCLIP targets of any SARS-CoV-2 protein (p<10¹³, KS test; FIG. 4G). Genes in the enriched GO processes, such as mitochondrial ATP synthesis and transport, protein N-linked glycosylation and COP II vesicle budding, are similarly upregulated by the overexpression of NSP12 (FIG. 4H). These observations provide support for the hypothesis that NSP12 has the capacity to increase the abundance of its eCLIP target RNAs.

To determine if SARS-CoV-2 proteins enhance the translation of endogenous genes, polysome profiling was performed on ORF9c, as it demonstrated the greatest ratio of changes in luciferase activity to changes in luciferase mRNA levels. First, the log 2 fold changes of polysomal mRNA levels versus total mRNA levels were determined in BEAS-2B cells overexpressing ORF9c and then compared it to wildtype BEAS-2B cells to obtain differential translation rates Δ log 2FoldChange. It was observed that the eCLIP targets of ORF9c have higher Δ log 2FoldChange in translation rates than genes that are not eCLIP targets of any SARS-CoV-2 protein (p<10-, KS test; FIG. 4I). Genes in the enriched pathways, such as protein processing in the ER, androgen receptor signaling, and protein N-linked glycosylation are similarly upregulated by the overexpression of ORF9c (FIG. 4J). Among the N-linked glycosylated GO term genes, Ribophorin I (RPN1) is part of an N-oligosaccharyl transferase complex that links high mannose oligosaccharides to asparagine residues found in the Asn-X-Ser/Thr consensus motif of nascent polypeptide chains, and UDP-Glucose Glycoprotein Glucosyltransferase 1 (UGGT1) is a soluble protein of the endoplasmic reticulum (ER) that selectively glucosylates unfolded glycoproteins. Represented in the mitochondrial ATP synthesis coupled electron transport and the respiratory electron transport chain GO processes, NDUFA4 is part of the enzyme cytochrome-c oxidase (or complex IV) and is important for its activity and biogenesis. Consistent with the data showing that exogenous expression of ORF9c can interact with RPNI, UGGT1 and NDUFA4 RNA and increase protein expression, it was found that SARS-CoV-2 infection increases RPNI, UGGT1 and NDUFA4 protein levels specifically in infected cells (FIGS. 4H-4I).

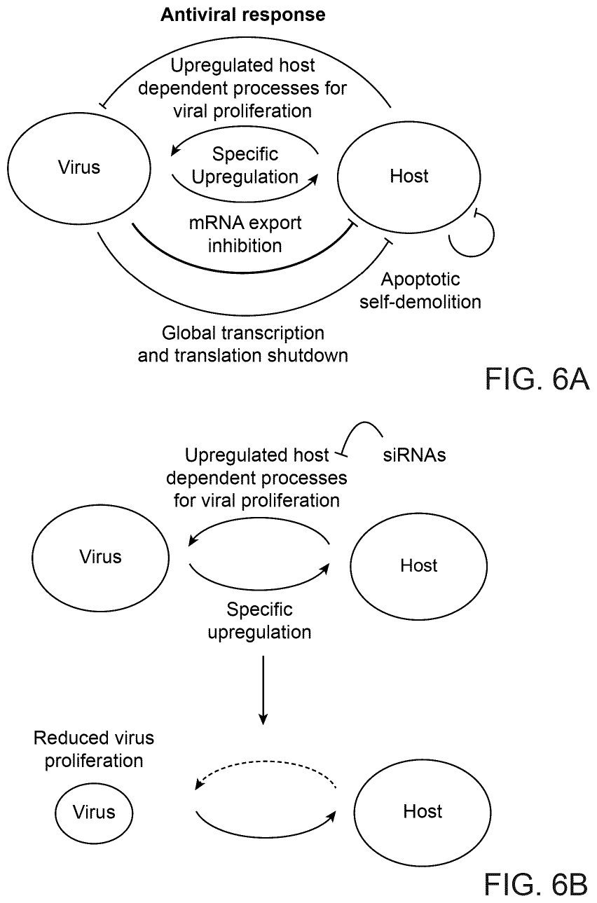

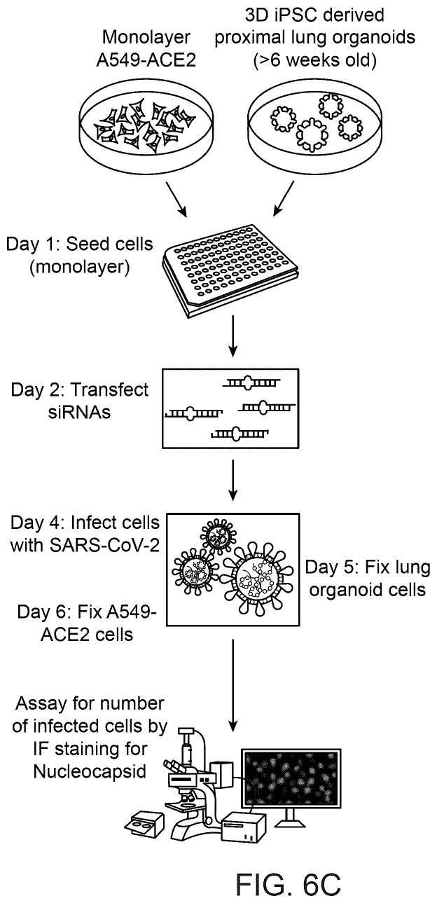

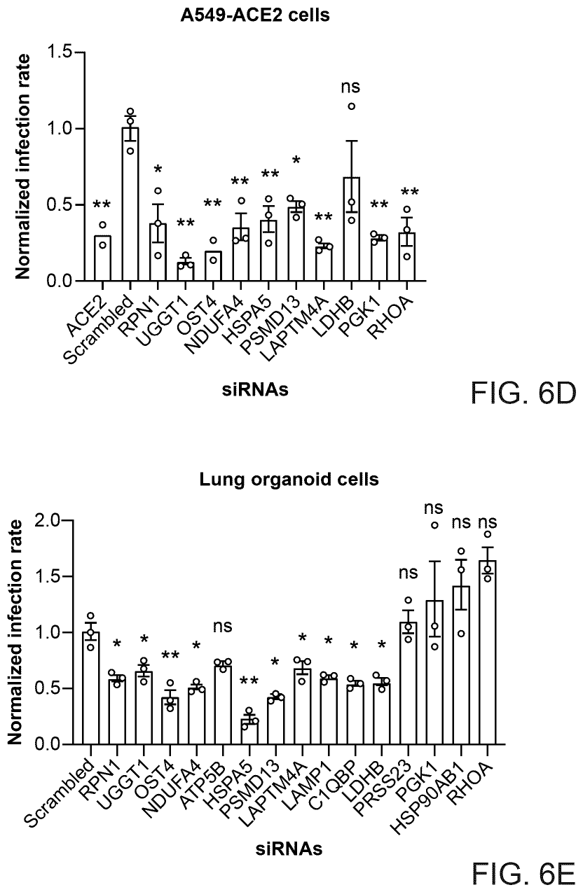

To determine if some of these host RNAs that interact with expression enhancing SARSCoV-2 proteins are pro-viral or antiviral, the impact of siRNA knockdown of these genes was investigated on viral infection or replication in human lung organoid cells (FIGS. 6A-6E). Human lung organoids are a physiologically relevant system to study infections and have been shown to be highly infectible by SARS-CoV-2. siRNAs were selected from the target mRNAs of SARSCoV-2 proteins with mRNA stabilization or translation enhancing activities, in addition to an anti-ACE2 siRNA and a scrambled sequence as a negative control.

Assays were performed for infected cells by immunofluorescence and infection rate was determined by measuring the total integrated fluorescence intensity of the stained nucleocapsid protein (FIG. 4K). To control for cell viability, the integrated intensity was divided to the area stained by DAPI, and the values were normalized to the scrambled control (FIG. 4L). It was found that siRNA knockdown of RPN1, UGGT1, NDUFA4, HSPA5, PSMD13, LAPTM4A, LAMP1, and LDHB significantly (p<0.05, two-tailed t-test) reduced infection rates for at least one of the tested SARS-CoV-2 variants compared to a scrambled siRNA control (FIG. 4L). Of note, siRNA knockdown of NDUFA4, UGGT1 and LAPTM4A significantly reduced viral infection in all three variants.

Taken together, these results suggest that SARS-CoV-2 proteins with a preference for binding to 5′ UTR and CDS regions have a capacity for increasing the abundance of target mRNAs and/or translation rates. Furthermore, it was found that eCLIP target genes are associated with enhanced RNA levels via NSP12 overexpression, and increased translation rates with ORF9c overexpression.

### Example 5—NSP9 Associates with the Nuclear Pore to Block mRNA Export

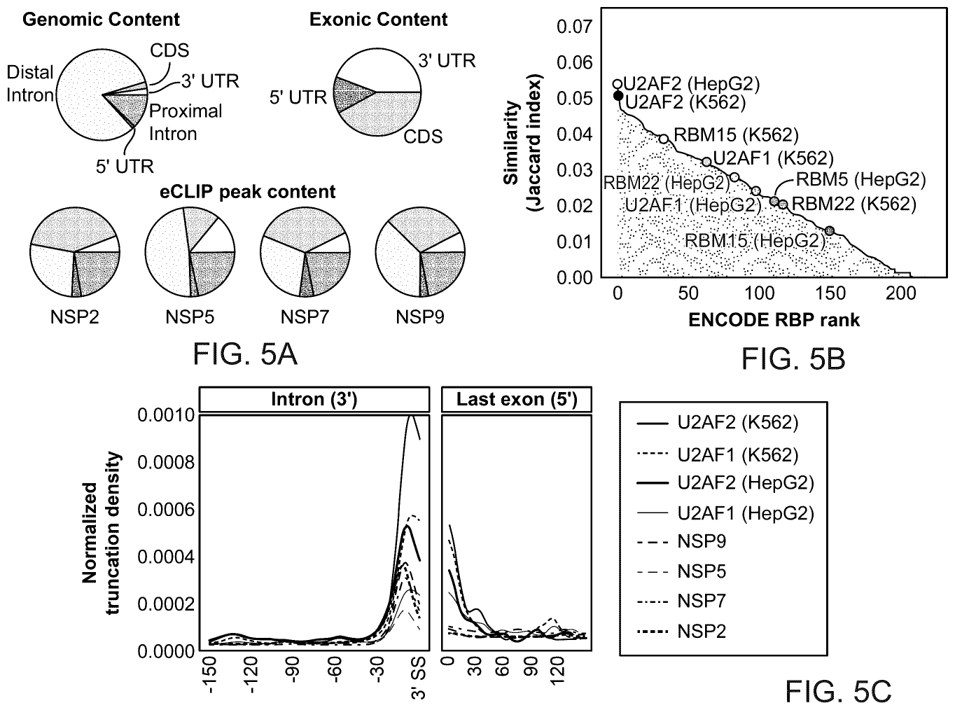

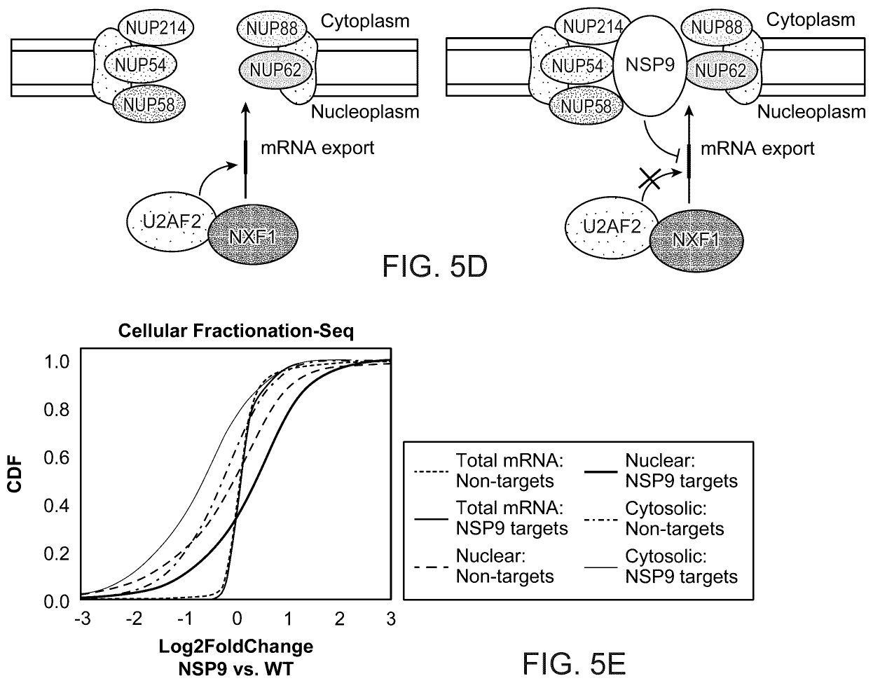

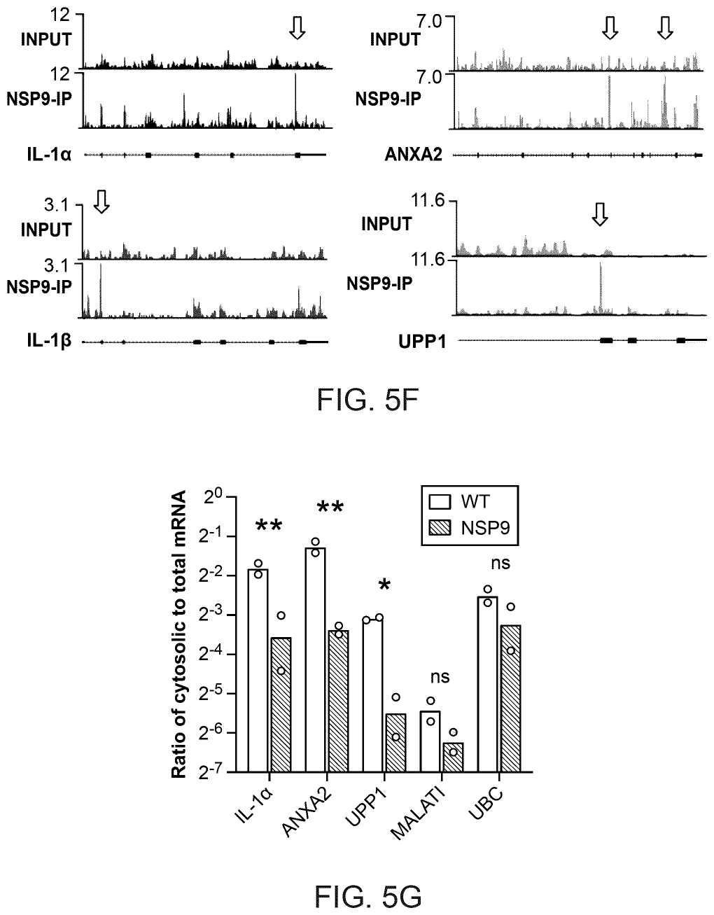

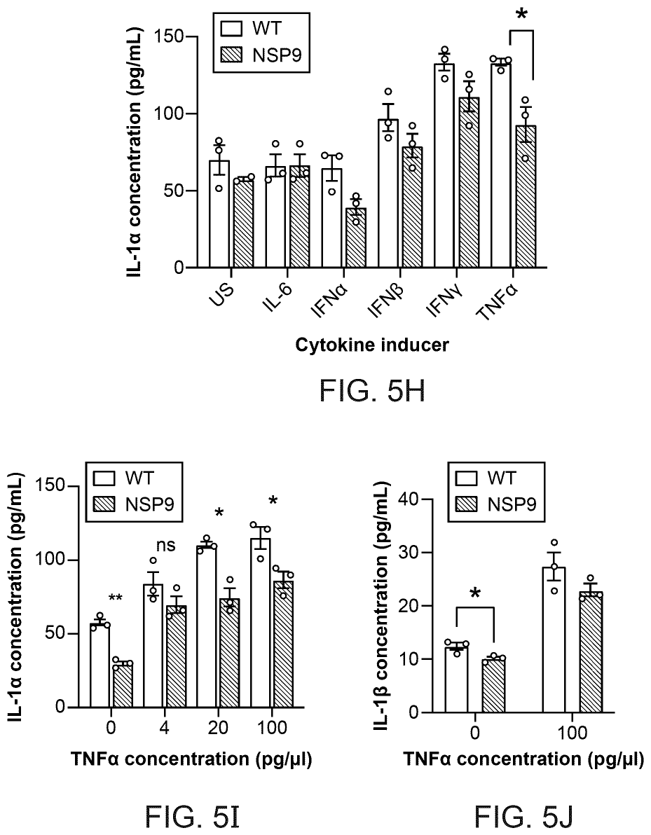

Since it was reported that several SARS-CoV-2 proteins are localized to the cell nucleus, it was found that the eCLIP peaks of NSP2, NSP5, NSP7, and NSP9 are enriched in intronic regions (FIG. 5A). To test whether these targets are implicated in infection induced alternative splicing, deep sequencing (>50 million 100 nt reads per sample) of SARS-CoV-2 infected A549-ACE2 cells was performed. A total of 1839 alternatively spliced genes were found across all five types of alternative splicing events i.e. alternative 5′ and 3′ splice site, skipped exons, skipped introns and mutually exclusive exons (false discovery rate <0.1, Inclusion level difference >0.05). By comparing genes with eCLIP peaks mapped to intronic regions or splice sites to genes not targeted by any of the SARS-CoV-2 proteins, observed no significant differences were observed in alternative splicing (significance level α=0.01, KS test). The little or no relationship with splicing led to consideration of other potential ways intronic binding by these SARS-CoV-2 proteins may be affecting the host transcriptome.

To infer molecular function, the similarity between each of 223 ENCODE RBP datasets were compared with NSP2, NSP5, NSP7, and NSP9 by computing the Jaccard Index of target genes. It was found that U2AF2's target gene set is most similar to NSP2, NSP7, and NSP9, and ranks highly for NSP5 (FIG. 5B). However, since the cell lines used for ENCODE—HepG2 and K562—and in this study—BEAS-2B—are different, the Jaccard indexes are low, at 0.050 for NSP7, 0.054 for NSP9, 0.057 for NSP5, and 0.074 for NSP2. To further ascertain similarity with U2AF2 protein-RNA interactions, the positional read density was inspected. The 5′ end of each eCLIP read can be used to approximate the crosslink site where reverse transcription is aborted or truncated when converting protein-bound RNA to cDNA. By taking the mean of the 5′ read truncation density across all target genes, a strong enrichment for the truncation site was observed at a median of 11 nt upstream of the 3′ splice site (FIG. 5C). Furthermore, a strong overlap was observed between U2AF2 eCLIP performed in both HepG2 and K562 cells with NSP2, NSP5, NSP7, and NSP9, with median truncation site at 10 nt upstream of 3′ splice site, providing evidence of substrate similarity. Using affinity mass-spectrometry, it was shown that NSP9 interacts with several nuclear pore complex proteins, including NUP62, NUP214, NUP88, NUP54 and NUP581 (FIG. 5D). It was confirmed that NUP62 indeed co-immunoprecipitated with NSP9. Even though U2AF2 was not found in the protein-protein interaction network of NSP9, it was previously reported to facilitate the binding of nuclear export factor TAP/NXF1 to its mRNA substrates. From these observations, it was hypothesized that NSP9 may interfere with mRNA export by associating with the nuclear pore and interfering with the U2AF/NXF1 complex for RNA substrate recognition (FIG. 5E). The significance of NSP2, NSP5, and NSP7 association with this intronic region may be less clear, and will benefit from future studies for clarification.

To determine if NSP9 inhibits mRNA export activity, assays were performed for the mRNA levels of NSP9 target genes in cytosolic and nuclear fractions. Both NSP9 expressing BEAS-2B cells and the wild type BEAS-2B cells were fractionated, followed by RNA extraction and preparation for mRNA sequencing. No difference was observed in log 2 fold changes of mRNA levels in NSP9 overexpressing cells versus wildtype cells between NSP9 eCLIP targets and non-targets, which agrees with the observation of lack of perturbation of target gene expression in the dual reporter assay. However, NSP9 eCLIP targets displayed greater log 2 fold change of mRNA levels in the nuclear fraction and lower levels in the cytosolic fraction than non-target genes (FIG. 5E). To validate the sequencing results, individual RT-qPCR were performed on the subcellular fractionated RNAs for individual target mRNAs IL-1α, ANXA2 and UPP1 (FIG. 5F), and lower cytosolic to total mRNA ratios were observed in NSP9-expressing versus parental cells, whereas the cytosolic mRNA levels of non-targeted controls MALAT1 and UBC were not significantly lowered (FIG. 5G). Even though nuclear RNA fractions were purified at high yields (>1 μg/μl), the RT-qPCR CT values of the target genes were too high (>25 cycles) for accurate quantification.

Interleukin 1α (IL-1α) and interleukin 1β (IL-1β) are important inflammatory cytokines constitutively produced in epithelial cells and plays a central role in regulating immune responses, including being a master cytokine in acute lung inflammation. To determine if NSP9 inhibiting the nucleocytoplasmic export of the mRNA of IL-1α has any impact on the production of this cytokine, an ELISA was performed on the growth media of BEAS-2B wild type and NSP9 expressing cells 48 hours after induction by several common cytokines. Interferon α, β and γ resulted in lowered IL-1α levels in NSP9 cells compared to wild type, though tumor necrosis factor alpha (TNFα) resulted in the greatest reduction (˜30%) (FIG. 5H). The observation of reduced IL-1α produced at different concentrations of TNFα (FIG. 5I) was reproduced. In addition, reduced IL-1β produced in NSP9 expressing cells than in wildtype BEAS-2B cells was observed (FIG. 5J). Thus, NSP9 association with the nuclear pore complex proteins aligns with the observation of decreased cytoplasmic abundance of NSP9 target mRNAs, suggesting that NSP9 interaction may directly inhibit nuclear export. Further, NSP9 reduced the production of its target gene IL-1α/β, which suggests that the export inhibition mechanism may be a strategy that SARSCoV-2 employs to dampen inflammatory host response.

Taken together, similarities in intronic protein-RNA interactions were observed by nonstructural proteins 2, 5, 7, and 9, which resembles the binding profile of splicing factor U2AF2.

It was further shown that NSP9 reduces cellular mRNA export, likely by interfering with U2AF/NXF1 substrate recognition. These findings suggest NSP9 may contribute to the viral effort in suppressing host gene expression.

1. A pharmaceutical composition comprising an inhibitory nucleic acid that is complementary to a portion of a target RNA associated with a gene selected from the group consisting of RPNI, UGGT1, OST4, NDUFA 4, HSPA5, PSMD13, LAPTM4A, LAMPI, CJOBP, and LDHB.

2.-9. (canceled)

10. The composition of claim 1, wherein the inhibitory nucleic acid comprises one or more small interfering RNAs (siRNA).

11. The composition of claim 10, wherein the one or more siRNAs comprise at least one sequence selected from the group consisting of SEQ ID NOs: 1-6.

12. The composition of claim 1, wherein the inhibitory nucleic acid comprises one or more antisense oligonucleotides.

13. The composition of claim 1, wherein the inhibitory nucleic acid comprises one or more locked nucleic acids (LNA).

14. The composition of claim 13, wherein the one or more LNAs comprises a sequence selected from the group consisting of SEQ ID NOs: 52 and 53.

15. The composition of claim 13, wherein the inhibitory nucleic acid comprises one or more phosphorothioate substitutions.

16. The composition of claim 13, wherein the inhibitory nucleic acid comprises one or more 2′-O-methyl modifications.

17. The composition of claim 13, wherein the inhibitory nucleic acid comprises one or more 2′-O-methoxyethyl modifications.

18. The composition of claim 13, wherein the inhibitory nucleic acid comprises one or more 2′-fluoro modifications.

19. The composition of claim 10, wherein the one or more siRNAs are a plurality, wherein each siRNA comprises the same sequence as the other siRNAs in the plurality.

20. The composition of claim 10, wherein the one or more siRNAs contain a mixture of siRNAs comprising at least two different sequences.

21. The composition of claim 13, wherein the LNA is enclosed within a vector.

22. The composition of claim 21, wherein the vector is a lentivirus.

23. The composition of claim 21, wherein the vector is an adenovirus.

24. The composition of claim 21, wherein the vector is an adeno-associated viral vector (AAV).

25. The composition of claim 21, wherein the vector is a liposome.

26. A method of treating Coronavirus disease 2019 (COVID-19) in a subject, the method comprising:
administering to the subject a therapeutically effective amount of the pharmaceutical composition of claim 1, thereby treating Coronavirus disease 2019 (COVID-19) in the subject.

27. The method of claim 26, wherein the therapeutically effective amount of the pharmaceutical composition decreases target RNA protein expression.

28. A method of reducing risk of SARS-Co V2 infection in a subject, the method comprising:
administering to the subject a therapeutically effective amount of the pharmaceutical composition of claim 1, thereby reducing risk of SARS-Co V2 infection in the subject.

29. (canceled)

30. (canceled)
